(19) United States
(12) Patent Application Publication
(10) Pub. No.: US 2024/0319209 A1
(43) Pub. Date: Sep. 26, 2024

(54) METHOD FOR QUANTIFYING AHSP PROTEIN AND ITS USE FOR PROGNOSING A HAEMOGLOBIN-RELATED DISORDER

(71) Applicants: INSTITUT NATIONAL DE LA SANTÉ ET DE LA RECHERCHE MÉDICALE, Paris (FR); ASSISTANCE PUBLIQUE-HÔPITAUX DE PARIS (APHP), Paris (FR); ETABLISSEMENT FRANÇAIS DU SANG (EFS), La Plaine Saint Denis (FR); UNIVERSITÉ PARIS-EST CRÉTEIL VAL DE MARNE, Creteil (FR)

(72) Inventors: Véronique BAUDIN-CREUZA, Créteil (FR); Frédéric GALACTEROS, Créteil (FR); Corinne VASSEUR, Créteil (FR)

(21) Appl. No.: 18/578,165

(22) PCT Filed: Jul. 15, 2022
(86) PCT No.: PCT/EP2022/069942

§ 371 Date: Jan. 10, 2024

(30) Foreign Application Priority Data
Jul. 16, 2021 (EP) 21306004.9

(51) Int. Cl. G01N 33/68; A61K 31/17; A61P 7/06

(52) U.S. Cl. CPC G01N 33/6893; A61K 31/17; A61P 7/06; G01N 2470/04; G01N 2800/22; G01N 2800/52

(57) ABSTRACT

The Alpha haemoglobin-stabilising protein (AHSP) is a key chaperone molecule synthesised in red blood cell (RBC) precursors. In this study, the inventors have developed a sandwich ELISA method which allows the quantification of AHSP protein in RBCs of normal subjects and in sickle cell anaemia (SCA) patients treated or untreated by hydroxycarbamide. AHSP was significantly more abundant in untreated SCA patients than in controls (p<0.0001). After hydroxycarbamide treatment, the AHSP decreased but remained significantly more abundant than in controls. A highly positive correlation was found between AHSP concentration and the α-Hb pool. So, like the α-Hb pool, the AHSP concentration could be a new and better RBC biomarker of SCA disease, much more easily measurable than the α-Hb pool. The AHSP ELISA dosage will facilitate the study of AHSP under different pathological conditions and could be a novel method of monitoring the AHSP translation regulation not only in RBCs but also in the different tissues where it is expressed. Thus, the present invention refers to a method for prognosing and/or diagnosing a haemoglobin-related disorder, such as sickle cell anaemia and beta-thalassaemia, in a subject in need thereof comprising measuring the level of AHSP by sandwich Elisa technique.

### FIELD OF THE INVENTION

The invention relates a method for determining the concentration of AHSP in a sample. The invention also relates to a method for prognosing a haemoglobin-related disorder, such as sickle cell anaemia, in a subject in need thereof. The invention also relates a method for monitoring a treatment against said haemoglobin-related disorder in a subject in need thereof.

### BACKGROUND OF THE INVENTION

Alpha haemoglobin-stabilising protein (AHSP) is a key chaperone molecule synthesised in red blood cell (RBC) precursors; it plays an important role in the formation of normal tetrameric haemoglobin (Hb). There are two genes for α-globin (HBA1 and HBA2), and therefore four α-globin genes in diploid cells (which can be represented as αα/αα). The coding sequences of HBA1 and HBA2 are identical. There are one gene for β-globin (HBB) and therefore two β-globin genes in diploid cells (which can be represented as β/β).

AHSP binds specifically to α-haemoglobin (α-Hb) to form a stable, but reversible, soluble complex that prevents freshly synthesised α-Hb chains from forming inclusion bodies, which might damage membrane structures and trigger apoptosis.^{1,2} For this reason, discovery of the AHSP gene rapidly led to predictions that its expression would modulate pathological states of α-Hb excess, such as beta-thalassaemia (β-thal).^{1,3} It was suggested that AHSP might act as a modifier gene, influencing phenotype, in thalassaemia patients.

Several studies have used assessments of mRNA levels in circulating reticulocytes based on real-time reverse transcription-quantitative polymerase chain reaction (RT-qPCR) to assess AHSP gene expression.⁴⁻⁶ Lai et al. reported large interindividual variations (by up to threefold) of AHSP expression between healthy individuals.⁴ They demonstrated that the level of AHSP expression was dependent on associations of several AHSP haplotypes linked to some clades. Lim et al. found that AHSP expression was significantly correlated with mean cell haemoglobin level, foetal Hb (HbF α₂γ₂) %, α-globin, β-globin and excess α-globin levels [α-(β+γ)] in individuals with HbE/β-thalassaemia. They concluded that AHSP might act in a secondary compensatory mechanism in RBCs, counterbalancing the excess α-globin chain in these patients.⁵ In 2015, Mahmoud et al. compared AHSP expression between patients with β-thal and sickle cell anaemia (SCA), another inherited haemoglobinopathy. They found higher levels of AHSP gene expression in SCA than in thalassaemia patients.⁶

Many studies based on AHSP gene expression analyses have identified AHSP as a potential biomarker of various diseases.^{1,3-5,7-10} However, despite these extensive studies of AHSP gene expression, only a few studies have directly determined the amounts of AHSP protein present in RBCs. One study based on western blotting indicated that there were at least 10⁷ AHSP molecules per late erythroid precursor, corresponding to a concentration of about 0.1 mM.¹ Another study, based on 2D-DIGE proteomics, showed that proteasomal subunits and chaperones, such as AHSP and heat shock protein 70, were upregulated in the cytosol of RBCs from patients with sickle cell disease (SCD) relative to control RBCs.¹¹ However, there is currently no biochemical method for the accurate quantification of AHSP in normal and pathological RBCs.

SCA is caused by the presence of abnormal Hb, HbS (α₂β^{S}₂, mutation of the HBB gene) in homozygous patients (SS patients), leading to polymerisation of HbS at a low oxygen concentration. The clinical expression of SCA is highly heterogeneous and there is broad inter- and intra-individual (during the patient's life) variability, with phenotypes ranging from mild to severe disease with significant organ damage and early death. Hydroxycarbamide (HC) has been shown to increase HbF levels, thereby decreasing the rate of HbS polymerisation. HC treatment is often the first choice in adult patients with symptomatic disease, to avoid transfusion and the risks it entails, and in children, to prevent further complications of the disease.¹² However, the response to HC is highly variable and differs from one patient to another.

β-thalassaemias are inherited autosomal recessive diseases characterised by a decrease or absent synthesis of the normal β-globin chain leading to an anemia of varying severity and severe disorders due to the less efficient erythropoiesis¹³. The severity of β-thalassaemia (β-thal) is directly correlated to the degree of imbalanced α/non α-globin chain synthesis and therefore the amount of unbound α-globin chain. Phenotypic diversity of β-thal depends on this imbalance which reflects all possible combinations of α- and β-globin genotypes, levels of HbF and co-inheritance of other modulating factors.

The inventors have previously demonstrated the presence of a soluble α-Hb pool in the RBC lysates of healthy subjects and β-thal patients¹⁴⁻¹⁵ They demonstrated that the soluble α-Hb pool in RBC lysates from β-thal intermediate patients was highly significant increased compared to control subjects and was a new marker to characterize these patients and may be clinically useful for characterizing and monitoring the evolution of the disequilibrium of globin synthesis in response to treatments. More recently, they showed the presence of a soluble α-Hb pool in the RBC lysates of SS patients¹⁶ which was reduced in SS patients under HC treatment¹⁷. We concluded that the soluble α-Hb pool could be a surrogate marker to monitor the HC response as well as to follow the patients' compliance to HC treatment. As it has been shown a progressive increase in AHSP gene expression following the expression of α-globin gene, during maturation of the RBC precursors¹⁸ the question arose whether there is also an AHSP pool in SS patients and particularly those who are under HC treatment.

Thus, the inventors have developed an enzyme-linked immunosorbent assay (ELISA) method to accurately quantify the amount of AHSP protein in normal and pathological RBCs. Then, the inventors investigated the presence of AHSP in RBCs of patients with SCA treated and untreated by HC or in RBCs of patient with β-thalassaemia in order to determine if AHSP could also be a surrogate marker of treatment monitoring.

### SUMMARY OF THE INVENTION

The inventors made the observation that it is possible to detect and quantify the free soluble AHSP protein in a blood sample. In a first aspect, the present invention relates to an in vitro method for determining the concentration of AHSP in a sample. In a second aspect, the invention refers to in vitro method for prognosing a haemoglobin-related disorder in a subject, and in particular for prognosing a sickle cell disease such as sickle cell anaemia or β-thalassaemia. In a third aspect, the invention refers to an in vitro method for monitoring haemoglobin-related disorder, such as sickle cell disease or beta-thalassaemia, in a subject in need thereof. In a fourth aspect, the invention refers to an in vitro method for monitoring treatment of haemoglobin-related disorder in a subject in need thereof.

In particular, the present invention is defined by the claims.

### DETAILED DESCRIPTION OF THE INVENTION

The inventors have developed a sandwich ELISA method which allows the quantification of the alpha haemoglobin-stabilising protein (AHSP) in RBCs of normal subjects and in sickle cell anaemia (SCA) patients treated or untreated by hydroxycarbamide (HC). They found that AHSP was significantly more abundant in untreated SCA patients than in controls (p<0.0001). After HC treatment, the AHSP decreased but remained significantly more abundant than in controls (p<0.0160). A highly positive correlation was found between AHSP concentration and the α-Hb pool, which is independent of the treatment. The AHSP concentration could be a new and better RBC biomarker of SCA disease, much more easily measurable than the α-Hb pool. The AHSP ELISA dosage will facilitate the study of AHSP under different pathological conditions and could be a novel method of monitoring the AHSP translation regulation not only in red blood cells (RBCs) but also in the different tissues where it is expressed.

### 1) Methods for Determining the AHSP Concentration

Herein, the inventors have developed a sandwich enzyme-linked immunosorbent assay (ELISA) method which allows the quantification of the AHSP in a sample.

As used herein, the terms “Alpha-Haemoglobin Stabilising Protein” or “AHSP” refers to a 102 amino acid protein which is highly conserved in humans, pigs, cows, and rats. AHSP is also sometimes referred to in the art as Erythroid Differentiation Related Factor (EDRF), or Erythroid Associated Factor (ERAF). Genbank accession number for AHSP includes Homo sapiens, Accession Number AF485325. AHSP binds specifically to α-haemoglobin (α-Hb) to form a stable, but reversible, soluble complex that prevents freshly synthesised α-Hb chains from forming inclusion bodies.

According to the invention, AHSP refers to free soluble AHSP (i.e not complexed to α-Hb).

As used herein, the term “α-Hb” refers to a 141 amino acid protein also called alpha haemoglobin, alpha globin chain or alpha chain. α-Hb protein corresponds to GenBank accession number NP_000549. Usually, in healthy subjects, adult human haemoglobin (Hb A) consists of four protein subunits, two subunits called alpha haemoglobin and two subunits called beta haemoglobin.

Accordingly, the invention also relates to an in vitro method for determining the concentration of AHSP in a sample comprising the steps of i) contacting said sample with two antibodies that specifically bind to AHSP, ii) measuring the amount of bound anti-AHSP antibody and iii) calculating the concentration of AHSP in the sample.

In some embodiments, the sample is previously obtained from a subject.

As used herein, and according to all aspects of the invention, the concentration of AHSP refers to the amount of the free soluble AHSP polypeptide in the sample, and more particularly in blood sample and more particularly in RBC.

According to the invention, an antibody that binds specifically to AHSP is an antibody that does not cross react with the AHSP/α-Hb complex or haemoglobin such as α-Hb and β-Hb.

Examples of said antibody include, but are not limited to, the biotin-conjugated polyclonal antibody anti-AHSP (reference ABIN2615200 of Antibodies-online Gmb) and the monoclonal anti-AHSP antibody (reference EPR12319 of Abcam).

In one embodiment, one antibody that binds to AHSP is the “capture” antibody, and is bound to a solid support, such as protein coupling or protein binding surface. Solid supports which can be used in the practice of the invention include substrates such as nitrocellulose (e. g., in membrane or microtiter well form); polyvinylchloride (e. g., sheets or microtiter wells); polystyrene latex (e.g., beads or microtiter plates); polyvinylidine fluoride; diazotized paper; nylon membranes; activated beads, magnetically responsive beads, and the like. As intended herein, immunoassays encompass those using a support selected in a group comprising beads (Luminex®, CBA®), a membrane (e.g. dot blot assays, Western blot assays, ELISPOT assays, etc), a high-binding ELISA plates (e.g. COSTAR High Binding ELISA Plates, Corning Incorporated, Corning, NY, USA).

In some embodiment, the capture antibody is a monoclonal anti-AHSP antibody.

In particular embodiment, the capture antibody is the monoclonal anti-AHSP antibody (reference EPR12319 of Abcam).

In one embodiment, the second antibody that specifically binds to AHSP is the “detecting” antibody. The detection is based on several different principles known in the art such as antibody recognition and signal amplification detect ion systems (for example, the biotin-streptavidin system). When a signal amplification system is used, the label includes the detecting antibody (such as anti-AHSP antibody conjugated with biotin), and a second protein, such as streptavidin, that is coupled with an enzyme. The enzyme produces a detectable, measurable signal via incubation with the appropriate substrate which is proportional to the concentration of AHSP. Examples of enzymes include, without limitation, alkaline phosphatase, amylase, luciferase, catalase, beta-galactosidase, glucose oxidase, glucose-6-phosphate dehydrogenase, hexokinase, horseradish peroxidase (HRP), lactamase, urease and malate dehydrogenase. Suitable substrates include, without limitation, TMB (3,3′,5,5′-tetramethyl benzidine), OPD (o-phenylene diamine), and ABTS (2,2′-azino-bis (3-ethylbenzthiozoline-6-sulfonic acid). The signal amplification approach may be used to significantly increase the assay sensitivity and high level reproducibility and performance.

In some embodiment, the second antibody that specifically binds to AHSP is coupled with biotin (“detecting antibody”).

In some embodiment, the detecting antibody is a biotin-conjugated anti-AHSP antibody. In particular embodiment, the detecting antibody is the biotin-conjugated polyclonal anti-AHSP antibody (reference ABIN2615200 of Antibodies-online Gmb).

In some embodiment, a sequential incubation of the two antibodies that bind to AHSP is performed.

In some embodiment, the sample is first contacted with the capture antibody, and then the detecting antibody is added.

In some embodiment, a protein, such as streptavidin, coupled with an enzyme, such as HRP, is added after the detecting antibody, wherein the protein binds to the detecting antibody.

In some embodiment, a substrate, such as TMB, to the enzyme is added after the protein. Thus, the invention relates to an in vitro method for determining the concentration of AHSP in a sample obtained from a subject, comprising the steps of i) contacting said sample with a capture antibody, ii) adding to said sample a detecting antibody, such as a biotin-conjugated anti AHSP antibody, iii) adding to said sample a protein binding to the detecting antibody, such as streptavidin, that is coupled with an enzyme, such as HRP, iv) adding a substrate, such as TMB, to the enzyme, and v) determining the concentration of AHSP in the sample.

In some embodiment, the capture antibody is previously bound to a solid support such as ELISA plate.

In some embodiment, the concentration of AHSP is determined via a detectable, measurable signal produced by adding a substrate, such as HRP, to the enzyme conjugated to the protein, such as streptavidin, binding to the detecting antibody, such as biotin-conjugated detecting antibody, wherein the substrate produces a signal proportional to the concentration of AHSP in the sample.

Thus, in particular, the invention relates to an in vitro method for determining the concentration of AHSP in a sample obtained from a subject, comprising the steps of i) contacting said sample with the capture antibody bound to a solid support, such as ELISA plate, ii) adding to said sample a detecting antibody, such as a biotin-conjugated anti AHSP antibody, iii) adding to said sample a protein binding, such as streptavidin, that is coupled with an enzyme, such as HRP, and iv) adding a substrate, such as TMB, to the enzyme such as HRP, conjugated to the protein, such as streptavidin, binding to the conjugated detecting antibody, such as biotin-conjugated detecting antibody, wherein the substrate produces a measurable signal proportional to the concentration of AHSP in the sample

According to the invention, the concentration of AHSP in a sample is determined from a standard curve generated with known concentrations of recombinant human AHSP.

In some embodiment, the standard curve refers to the detectable, measurable signal (i.e absorbance) produced by adding a substrate, such as TMB, to the enzyme, such as HRP, conjugated to the protein, such as streptavidin, binding to the conjugated detecting antibody, such as biotin-conjugated detecting antibody as a function of the concentration of human recombinant AHSP.

In some embodiment, the human recombinant AHSP can be expressed with gene fusion expression systems and produced by genetic engineering as a fusion protein. Example of gene fusion expression systems include thioredoxin, Glutathione S Transferase (GST), Maltose Binding Protein (MBP), Green Fluorescent Protein (GFP), Yellow Fluorescent protein (YFP), intein, NusA or luciferase as fusion proteins. The human recombinant AHSP can also be expressed with the polypeptide protein Tag such as polyhistidine Tag, Strep-Tag, FLAG-Tag, S-Tag, Dsb A Tag, Dsb C tag, hemagglutinin Tag (HA-Tag) or myc-Tag.

In some embodiment, the recombinant human AHSP is produced as a fusion protein with glutathione S-transferase (GST-AHSP) and is purified after cleavage of the GST moiety.

In preferred embodiment, the recombinant human AHSP is produced as a fusion protein with glutathione S-transferase (GST-AHSP) in E. coli cells containing the pGEX-AHSP expression plasmid, and is purified after cleavage of the GST moiety by enzyme, such as thrombin or PreScission Protease™, as previously described in Brillet T, et al,. J Biol Chem. 2010¹⁹; and Baudin-Creuza, et al,. J Biol Chem. 2004²⁰.

The method for determining the concentration of AHSP according to the invention can also be used in numerous applications, such as:
prognosing haemoglobin-related disorder
monitoring of treatment of haemoglobin-related disorder,
drug screening,
studying haemoglobin-related disorder,
studying mutation of AHSP
monitoring the regulation of AHSP translation.
   2) Methods for Diagnosis and/or Prognosis of the Invention

Accordingly, the invention relates to an in vitro method for diagnosis and/or prognosis haemoglobin-related disorder in a subject, comprising the steps of i) determining the concentration of alpha-haemoglobin stabilising protein (AHSP) in a sample obtained from the subject, and ii) positively correlating said concentration of AHSP with the diagnosis and/or the prognosis of a haemoglobin-related disorder in said subject.

In other words, the invention relates to an in vitro method for prognosis haemoglobin-related disorder in a subject, comprising the steps of i) determining the concentration of alpha-haemoglobin stabilising protein (AHSP) in a sample obtained from the subject, ii) comparing the concentration of AHSP determined at step i) with a reference value, and iii) concluding that the subject has a haemoglobin-related disorder when the concentration of AHSP determined at step i) are higher than the reference value.

Accordingly, the invention relates to an in vitro method for diagnosis haemoglobin-related disorder in a subject, comprising the steps of i) determining the concentration of alpha-haemoglobin stabilising protein (AHSP) in a sample obtained from the subject, ii) comparing the concentration of AHSP determined at step i) with a reference value, and iii) concluding that the subject has a haemoglobin-related disorder when the concentration of AHSP determined at step i) are higher than the reference value.

In other words, the invention relates to an in vitro method for assessing a subject's risk of having haemoglobin-related disorder, comprising the steps of i) determining the concentration of alpha-haemoglobin stabilising protein (AHSP) in a sample obtained from the subject, ii) comparing the concentration of AHSP determined at step i) with a reference value, and iii) concluding that the subject has a high risk of having haemoglobin-related disorder when the concentration of AHSP determined at step i) are higher than the reference value.

As used herein and according to all aspects of the invention, the concentration of AHSP refers to the amount of the free soluble AHSP polypeptide in blood sample, and more particularly in RBC.

As used herein and according to all aspects of the invention, the term “sample” denotes blood, fresh whole blood, peripheral-blood, cord blood, red blood cell sample.

In particular embodiment, the sample is a blood sample, and in more particular embodiment the sample is RBC. In some embodiment, the sample is RBC lysates, also named haemolysate. In some embodiment, the sample is reticulocytes.

As used herein, the term “red blood cells” or “RBCs”, also called as red cells, red blood corpuscles, erythroid cells, including erythrocytes and reticulocytes are the most common type of blood cell and the vertebrate's principal means of delivering oxygen to the body tissues via blood flow through the circulatory system. RBCs take up oxygen in the lungs, and release it into tissues while squeezing through the body's capillaries. The cytoplasm of RBC is rich in haemoglobin, an iron-containing biomolecule that can bind oxygen and is responsible for the red color of the cells and the blood.

As used herein, the term “RBC lysates” refers to RBCs including erythrocytes and reticulocytes which are lysed for example with four volumes of cold distilled water. The mixture was incubated for 30 min on ice, centrifuged at 16,000×g for 30 minutes at +4° C. and RBC lysates or haemolysate were recovered in the supernatant and immediately frozen at −80° C.

As used herein, the term “subject” or “patient” refers to any mammal, such as a rodent, a feline, a canine, and a primate. Particularly, in the present invention, the term “subject” refers to a human and more particular, to a human afflicted with haemoglobin-related disorder. In some embodiment, the subject is a human afflicted with sickle cell disease and more particular with sickle cell anaemia. In some embodiment, the subject is a human afflicted with beta-thalassaemia.

“Risk” in the context of the present invention, relates to the probability that the patients have or not an haemoglobin-related disorder. Risk can be measured with reference to either actual observation post-measurement for the relevant cohort (i.e validation of the haemoglobin-related disorder with further measurement such as microcytic hypochromic anemia, nucleated red blood cells on peripheral blood smear, . . . ), or with reference to index values developed from statistically valid historical cohorts._Odds ratios, the proportion of positive events (i.e having haemoglobin disorder) to negative events (i.e not having haemoglobin disorder) for a given test result, are also commonly used (odds are according to the formula p/(1−p) where p is the probability of having haemoglobin-disorder and (1−p) is the probability of no having haemoglobin disorder).

As used herein, the term “haemoglobin-related disorder” also known as “haemoglobinopathies” has its general meaning in the art and refers to a group of inherited autosomal recessive pathologies affecting the Hb. Various abnormalities can result from changes (mutations or deletions) in the genes that regulate the production of Hb. Other mutations, even if they do not prevent the production of adequate amounts of Hb chains, cause an alteration in their structure. They are known as haemoglobin variants and they alter the function and/or the stability of the entire Hb molecule.

These diseases are characterized by an excess or a deficit in α-Hb and/or β-Hb and/or AHSP. Haemoglobin-related disorder includes but are not limited to haemoglobinopathy such as thalassaemia (such as α-Thalassaemia or β-Thalassaemia), Haemoglobin C disease, Haemoglobin S disease, Haemoglobin SC disease, Haemoglobin E disorders (or Hb E related syndroms), unstable abnormal Hb (such as Hb Taybe) and sickle cell diseases; myelodysplasia such as refractory anaemia, refractory cytopenia with multilineage dysphasia and mixed myelodysplastic/myeloproliferative neoplasms. Haemoglobin variant and mutation that cause haemoglobin-related disorder are well known in the art and are disclosed in Giardine et al, Nucleic Acids Res. 2014 January; 42; and on https://globin.bx.psu.edu/hbvar/menu.htm²¹.

Patients affected with haemoglobin-related disorder may have ineffective erythropoiesis.

The method according to the invention is also useful for diagnosing and/or prognosis and/or monitoring the erythropoiesis in a subject.

Thus, the invention relates to an in vitro method for diagnosis and/or prognosis and/or monitoring erythropoiesis in a subject, comprising the steps of i) determining the concentration of alpha-haemoglobin stabilising protein (AHSP) in a sample obtained from the subject, and ii) positively correlating said concentration of AHSP with the diagnosis and/or the monitoring of an ineffective erythropoiesis in said subject.

In some embodiments, regarding the methods for prognosis/diagnosis/asses the risk of having haemoglobin-related disorder of the invention, the haemoglobin-related disorder is selected among sickle cell disease (SCD) and beta-thalassaemia.

Thus, the invention refers to an in vitro method for diagnosis haemoglobin-related disorder in a subject, comprising the steps of i) determining the concentration of alpha-haemoglobin stabilising protein (AHSP) in a sample obtained from the subject, ii) comparing the concentration of AHSP determined at step i) with a reference value, and iii) concluding that the subject has a haemoglobin-related disorder when the concentration of AHSP determined at step i) are higher than the reference value, wherein the haemoglobin-related disorder is sickle cell disease or beta-thalassaemia and wherein the reference value is determined from the concentration of AHSP from one or more healthy subject (i.e that has not been diagnosed for sickle cell disease or beta-thalassaemia).

In some embodiments, the haemoglobin-related disorder is sickle cell disease (SCD).

As used herein, the term “sickle cell disease” or “SCD” has its general meaning in the art and refers to an inherited blood disorder caused by a genetic mutation in the β-chains of haemoglobin, due to mutations in the HBB gene on chromosome 11 leading to the presence of abnormal HbS. The clinical expression of SCD is highly heterogeneous and there is broad inter- and intra-individual (during the patient's life) variability, with phenotypes ranging from mild to severe disease with significant organ damage and early death. In SCD, these sickle cells also become rigid and sticky, which can slow or block blood flow results in a risk of various life-threatening complications. The term includes sickle cell anaemia (SCA) (haemoglobin SS or homozygous SS patient), haemoglobin SC disease and haemoglobin S/beta-thalassaemia.

In some embodiment, the haemoglobin-related disorder is sickle cell anaemia (SCA).

Thus, in particular, the invention refers to an in vitro method for prognosing sickle cell disease in a subject, comprising the steps of i) determining in a sample obtained from the subject the concentration of AHSP, ii) comparing the concentration of AHSP determined at step i) with a reference value; and iii) concluding that the subject has sickle cell disease when the concentration of AHSP determined at step i) are higher than the reference value.

In some embodiments, as illustrated in the experimental study, it is concluded that the subject has sickle cell disease when the concentration of AHSP determined at step i) is between 1.5 to 4 factor higher than the reference value, wherein the reference value is determined from the concentration of AHSP from one or more healthy subject (i.e that has not been diagnosed for a haemoglobin-related disorder).

In particular embodiments, as illustrated in the experimental study with an immunodetection assay such ELISA assay (see example 1), it is concluded that the subject has sickle cell disease when the concentration of AHSP determined at step i) is higher than the reference value and lower than 4 μg/ml, wherein the reference value is determined from the concentration of AHSP from one or more healthy subject (i.e that has not been diagnosed for a haemoglobin-related disorder).

Thus, in particular embodiment, the invention refers to an in vitro method for prognosing and/or diagnosing sickle cell disease in a subject, comprising the steps of i) determining in a sample obtained from the subject the concentration of AHSP, ii) comparing the concentration of AHSP determined at step i) with a reference value; and iii) concluding that the subject has sickle cell disease when the concentration of AHSP determined at step i) is higher than the reference value and lower than 4 μg/ml.

In some embodiments, the haemoglobin-related disorder is beta-thalassaemia.

Thus, in particular, the invention refers to an in vitro method for diagnosing or prognosing beta-thalassemia in a subject, comprising the steps of i) determining in a sample obtained from the subject the concentration of AHSP, ii) comparing the concentration of AHSP determined at step i) with a reference value; and iii) concluding that the subject has beta-thalassaemia when the concentration of AHSP determined at step i) are higher than the reference value.

In some embodiments, as illustrated in the experimental study with immunodetection assay such ELISA assay (see example 2), it is concluded that the subject has beta-thalassaemia when the concentration of AHSP determined at step i) is at least 6, 7, 8, 9, 10, 20, 30, or 40 factor higher than the reference value, wherein the reference value is determined from the concentration of AHSP from one or more healthy subject (i.e that has not been diagnosed for a haemoglobin-related disorder).

In particular embodiment, as illustrated in the experimental study with immunodetection assay such ELISA assay (see example 2), it is concluded that the subject has beta-thalassaemia when the concentration of AHSP determined at step i) is higher than 7 μg/ml.

Thus, in particular embodiment, the invention refers to an in vitro method for prognosing and/or diagnosing beta-thalassaemia in a subject, comprising the steps of i) determining in a sample obtained from the subject the concentration of AHSP; and ii) concluding that the subject has beta-thalassaemia when the concentration of AHSP determined at step i) is higher than 7 μg/ml.

In particular embodiment, significantly higher concentration of AHSP positively correlates to the severity of beta-thalassaemia in said subject.

In particular embodiment, significantly higher concentration of AHSP as compared to reference value correlates to the severity of beta-thalassaemia in said subject.

Thus, in particular embodiment, the invention refers to an in vitro method for staging or diagnosing beta-thalassaemia comprising i) determining in a sample obtained from the subject the concentration of AHSP, ii) comparing the concentration of AHSP determined at step i) with a reference value and iii) correlating said concentration of AHSP with the staging or diagnosing of beta-thalassaemia, wherein significantly higher concentration of AHSP as compared to reference value correlates to the severity of beta-thalassaemia in said subject

In particular embodiment, the invention refers to an in vitro method for stratifying or diagnosing haemoglobin-related disorder selected from the group consisting in sickle-cell disease and beta-thalassaemia in a subject, comprising the steps of i) determining in a sample obtained from the subject the concentration of AHSP, ii) comparing the concentration of AHSP determined at step i) with a reference value; and iii) concluding that the subject has sickle cell disease when the concentration of AHSP determined at step i) are higher than the reference value and lower than 4 μg/ml or concluding that the subject has beta-thalassaemia when the concentration of AHSP determined at step i) are higher than 7 μg/ml.

As used herein, the term “thalassaemia” has its general meaning in the art and refers to to inherited autosomal recessive diseases characterized by decreased haemoglobin production. According to the invention, thalassaemia includes alpha-thalassaemia, beta-thalassaemia and delta-thalassaemia.

As used herein, the term “beta thalassaemia” or “β-thalassaemia” has its general meaning in the art and refers to a group of inherited blood diseases characterized by a quantitative deficiency of β-globin chains underlaid by a heterogeneity of molecular defects in HBB gene on chromosome 11. Consequences include anaemia, of different severity according to the mutations involved, several other severe disorders due to the increase in medullar erythropoiesis, impaired statural growth and bone structure, accelerated iron turnover, and haem catabolism, and their own clinical consequences. Beta-thalassemia is characterized by a total (β0) or partial (β+) deficiency in the synthesis of the β-globin chains of Hb.

The severity of the disease depends on the nature of the mutation and on the presence of mutations in one or both alleles. β thalassaemia major (Mediterranean anemia or Cooley anemia) is caused by a β0/β0 genotype. No β chains are produced, and thus no HbA can be assembled. This is the most severe form of β-thalassaemia.

β thalassaemia intermedia is caused by a β+/β0 or β+/β+ genotype. In this form, some haemoglobin A is produced. β thalassaemia minor is caused by a β0/β0 or β/β+ genotype. Only one of the two β globin alleles contains a mutation, so β chain production is not severely compromised and patients may be relatively asymptomatic. Several mutations in the β-globin gene cluster, such as those that occur in β-thalassemic individuals, can alter α/β-globin chain balance leading to accumulation and precipitation of free α-globin chains.

In some embodiments, the reference value is a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognised by a person of ordinary skilled in the art. For example, retrospective measurement of the AHSP concentration in properly banked historical subject samples may be used in establishing the reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. The full name of ROC curve is receiver operator characteristic curve, which is also known as receiver operation characteristic curve. It is mainly used for clinical biochemical diagnostic or prognostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), Stata/Se version 12.0 software (StataCorp LP, College Station, TX, USA), etc.

In some embodiments, the reference value is the concentration of AHSP in a healthy subject (i.e that has not been diagnosed for a haemoglobin-related disorder).

In some embodiments, the reference value is determined from the concentration of AHSP from one or more healthy subject (i.e that has not been diagnosed for a haemoglobin-related disorder).

Furthermore, retrospective measurement of the level of the markers of the invention in properly banked historical subject samples may be used in establishing these reference values.

In some embodiments, the reference value is the concentration of AHSP detected in previous samples obtained from the subject. In some embodiment, the reference value is the highest concentration of AHSP determined in healthy subjects (i.e that has not been diagnosed for a haemoglobin-related disorder).

In some embodiments, the reference value is ranging from 0.4 to 1.48 μg/mL, and more particularly from 0.87 to 1.22 μg/mL.

In some embodiments, as illustrated in the experimental study with an immunodetection assay such ELISA assay, the reference value is selected from the value of control group in table 2.

In some embodiments, as illustrated in the experimental study with an immunodetection assay such ELISA assay, the reference value is 0.4, 0.42, 0.5, 0.58, 0.6, 0.61, 0.7, 0.75, 0.77, 0.8, 0.82, 0.85, 0.87, 0.9, 0.91, 0.92, 0.95, 0.96, 1.00, 1.03, 1.05, 1.10, 1.12, 1.15, 1.2 1.22, μg/mL.

In some embodiments as illustrated in the experimental study with an immunodetection assay such ELISA assay, the reference value is 0.82 or 1.22 μg/mL. In some embodiments, a reference value may be determined for each haemoglobin-related disorder.

In some embodiments, as illustrated in the experimental study with an immunodetection assay such ELISA assay, when the method for diagnosis, staging or prognosing beta-thalassaemia, the reference value is 7 μg/ml.

Typically, AHSP concentration may be determined for example by capillary electrophoresis-mass spectroscopy technique (CE-MS), flow cytometry, mass cytometry or immunoassay such as an enzyme-linked immunosorbent assay (ELISA), performed on the sample.

In some embodiment, the AHSP concentration is determined by immunoassay.

As used herein, the term “Immunoassays” encompass any assay wherein a capture reagent (i.e binding partner) is immobilised on a support and wherein detection of an analyte of interest (i.e AHSP) is performed through the use of antibodies directed against the said analyte of interest (i.e AHSP). Such assays include, but are not limited to agglutination tests; enzyme-labeled and mediated immunoassays, such as enzyme-linked immunosorbent assays (ELISAs); biotin/avidin type assays; radioimmunoassays; immunoelectrophoresis; immunoprecipitation, capillary electrophoresis-mass spectroscopy technique (CE-MS) etc. The reactions generally include revealing labels such as fluorescent, chemiluminescent, radioactive, enzymatic labels or dye molecules, or other methods for detecting the formation of a complex between the antigen and the antibody or antibodies reacted therewith. Immunoassays includes competition, direct reaction, or sandwich type assays.

In some embodiment, the AHSP concentration is determined by enzyme-labeled and mediated immunoassays (ELISA).

In some embodiment, the AHSP concentration is determined by direct ELISA. The AHSP is directly immobilized to a surface of a multi-well plate and detected with a biotin-conjugated detection antibody specific for the AHSP. This antibody is directly conjugated to a detection systems (horseradish peroxidase (HRP)-conjugated Streptavidin or other detection molecules).

In some embodiment, the AHSP concentration is determined by indirect ELISA. The AHSP is directly immobilized to a surface of a multi-well plate and detected with an unconjugated primary detection antibody specific for the AHSP. A conjugated secondary antibody directed against the host species of the primary antibody is then added. Substrate then produces a signal proportional to the amount of AHSP bound in the well.

In some embodiment, the AHSP concentration is determined by sandwich ELISA.

According to the invention, “sandwich” ELISA refers to an immunoassay wherein free AHSP may be sandwiched between two antibodies that specifically bind to free AHSP.

Accordingly, the invention relates to a method for prognosing haemoglobin-related disorder in a subject, comprising the steps of i) determining in a sample obtained from the subject the concentration of AHSP by an enzyme-labeled and mediated immunoassays, ii) comparing the concentration of AHSP determined at step i) with a reference value, and iii) concluding that the subject has a haemoglobin-related disorder when the concentration of AHSP determined at step i) are significantly higher than the reference value.

In some embodiment, the concentration of AHSP in a sample is determined by the method for determining the concentration of AHSP according to the invention.

Thus, the invention relates to a method for diagnosing or prognosing a haemoglobin-related disorder in a subject, comprising the steps of i) contacting said sample with a capture antibody bound to a solid support, ii) adding to said sample a detecting antibody, iii) adding to said sample a protein binding, such as streptavidin, that is coupled with an enzyme, such as HRP, iv) adding a substrate, such as TMB, to the enzyme, v) determining the concentration of AHSP in the sample, vi) comparing the concentration of AHSP determined at step v) with a reference value, and vii) concluding that the subject has a haemoglobin-related disorder when the concentration of AHSP determined at step v) are higher than the reference value.

Thus, the invention relates to a method for assessing a subject's risk of having haemoglobin-related disorder, comprising the steps of i) contacting said sample with a capture antibody bound to a solid support, ii) adding to said sample a detecting antibody, iii) adding to said sample a protein binding, such as streptavidin, that is coupled with an enzyme, such as HRP, iv) adding a substrate, such as TMB, to the enzyme, v) determining the concentration of AHSP in the sample, vi) comparing the concentration of AHSP determined at step v) with a reference value, and vii) concluding that the subject has a high risk of having haemoglobin-related disorder when the concentration of AHSP determined at step v) are higher than the reference value.

In some embodiments, all the methods of the invention are performed in vitro or ex vivo.

### 3) Method of Stratification

The inventors demonstrated that the concentration of free AHSP in the f-thal patients haemolysates was greatly increased compared to both control subjects and SCA patients (FIG. 4).

Accordingly, the invention refers also to a method for classifying and/or stratifying haemoglobin-related disorder selected from the group consisting in sickle-cell disease and beta-thalassaemia comprising the steps of: i) determining the concentration of alpha-haemoglobin stabilising protein (AHSP) in a sample obtained from the subject, ii) comparing the concentration of AHSP determined at step i) with a reference value, and iii) concluding that the subject has a sickle cell disease when the concentration of AHSP determined at step i) are higher by a factor between 1.5 and 4 than the reference value or concluding that the subject has a β-thalassaemia when the concentration of AHSP determined at step i) are at least 6 factor higher than the reference value.

In particular embodiment, regarding the method for classifying and/or stratifying, the reference value is determined from the concentration of AHSP from one or more healthy subject (i.e that has not been diagnosed for a haemoglobin-related disorder).

In other words, the invention refers also to a method for classifying and/or stratifying haemoglobin-related disorder selected from the group consisting in sickle-cell disease and beta-thalassaemia comprising the steps of i) determining the concentration of alpha-haemoglobin stabilising protein (AHSP) in a sample obtained from the subject, ii) comparing the concentration of AHSP determined at step i) with a reference value, and iii) concluding that the subject has a sickle cell disease when the concentration of AHSP determined at step i) than the reference value and lower than 4 μg/ml or concluding that the subject has a thalassaemia when the concentration of AHSP determined at step i) are higher than 7 μg/ml.

### 4) Methods for Monitoring Haemoglobin-Related Disorder and its Treatment of the Invention

An additional object of the invention relates to an in vitro method for monitoring haemoglobin-related disorder in a subject in need thereof comprising the steps of i) determining the concentration of AHSP in a sample obtained from the subject at a first specific time, ii) determining the concentration of AHSP in a sample obtained from the subject at a second specific time, iii) comparing the concentration determined at step i) with the concentration determined at step ii), and iv) concluding that the haemoglobin-related disorder has evolved in worse manner when the concentration determined at step ii) is higher than the concentration determined at step i).

In some embodiments, the concentration of AHSP determined at the step i) is determined when the subject is in steady state.

As used herein, subjects are considered to be in a steady state at least 8 to 10 weeks after, or two weeks before any vaso-occlusive crisis or other clinical event resulting in hospitalisation.

In some embodiments, regarding the methods of the invention for monitoring the haemoglobin-related disorder, the haemoglobin-related disorder is sickle-cell disease or beta-thalassaemia.

In some embodiments, the haemoglobin-related disorder is sickle cell disease such as sickle cell anaemia.

In some embodiments, the concentration of AHSP in a sample is determined by the method for determining the concentration of AHSP according to the invention.

The inventors found also that the concentration of AHSP decreased after treatment in SCA patients but remained significantly more abundant than in controls (i.e healthy subjects).

Accordingly, in another object, the invention relates to an in vitro method for monitoring the treatment of haemoglobin-related disorder in a subject in need thereof, comprising the steps of i) determining the concentration of AHSP in a sample obtained from the subject at a first specific time, ii) determining the concentration of AHSP in a sample obtained from the subject at a second specific time, iii) comparing the concentration determined at step i) with the concentration determined at step ii), and iv) concluding that said treatment is efficient when the concentration determined at step ii) is lower than the concentration determined at step i).

In some embodiments, the concentration of AHSP determined at the step i) is determined when the subject is in steady state.

In some embodiments, the first-time specific time where the concentration of AHSP is determined at step i) is before or at the beginning of said treatment.

Thus, in some embodiments, the invention relates to an in vitro method for monitoring the treatment of haemoglobin-related disorder in a subject in need thereof, comprising the steps of i) determining the concentration of AHSP in a sample obtained from the subject before or at the beginning of said treatment, ii) determining the concentration of AHSP in a sample obtained from the same subject at a second specific time of the treatment, iii) comparing the concentration of AHSP determined at step ii) with the concentration of AHSP determined at step i), and iv) concluding that said treatment is efficient when the concentration determined at step ii) is lower than the concentration of AHSP determined at step i).

In some embodiments, regarding the methods of the invention for monitoring the treatment, the haemoglobin-related disorder is sickle-cell disease or beta-thalassaemia.

In some embodiments, the second-time specific time where the concentration of AHSP is determined at step ii) is ranging between ten days and eighteen months after the start of said treatment.

In some embodiments, the second-time specific time of the disease where the concentration of AHSP is determined at step ii) is 10, 11, 12, 13, 14, 15, 16, 17, 18, 19, 20, 21, 22, 23, 24, 25, 26, 27, 28, 29, or 30 days, or 1, 2, 3, 4, 5, 6, 7, 8, 9, 10, 11, 12, 13, 14, 15, 16, 17 or 18 months after the start of said treatment.

In some embodiments, when it is concluded that the treatment is efficient, the treatment is continued.

Thus, the invention refer to a method for treating or monitoring haemoglobin-related disorder in subject in need comprising a step of i) performing the in vitro method for monitoring the treatment of haemoglobin-related disorder according to the invention and ii) continuing the treatment of haemoglobin-related disorder if it is concluded that said treatment is efficient. In particular embodiment, regarding the methods for treating or monitoring the treatment of the invention, the haemoglobin-related disorder is sickle-cell disease or beta-thalassaemia.

The invention of the method can also be used to monitoring the end of treatment.

Accordingly, in another aspect, the invention relates to an in vitro method for monitoring the interruption of the treatment of haemoglobin-related disorder in a subject in need thereof, comprising the steps of i) determining the concentration of AHSP in a sample obtained from the subject treated with said treatment, ii) determining the concentration of AHSP in a sample obtained from the same subject at a specific time after the interruption of said treatment, iii) comparing the concentration of AHSP determined at step ii) with the concentration of AHSP determined at step i), and iv) concluding that said treatment exhibit not effect anymore when the concentration of AHSP determined in step ii) is higher than the concentration of AHSP determined in step i).

In some embodiments, it is concluded that said treatment exhibit not effect anymore when the concentration of AHSP determined in step ii) is similar than the concentration of AHSP determined in step i).

In some embodiments, the second-time specific time where the concentration of AHSP is determined at step ii) is ranging between ten days and eighteen months after the interruption of said treatment.

In some embodiments, the second-time specific time of the disease where the concentration of AHSP is determined at step ii) is 10, 11, 12, 13, 14, 15, 16, 17, 18, 19, 20, 21, 22, 23, 24, 25, 26, 27, 28, 29, or 30 days or 1, 2, 3, 4, 5, 6, 7, 8, 9, 10, 11, 12, 13, 14, 15, 16, 17 or 18 months after the interruption of treatment.

In some embodiments, when it is concluded that the treatment exhibits not effect anymore or is not efficient, the treatment is adapted/modified.

Thus, the invention refer to a method for treating haemoglobin-related disorder or monitoring the treatment of haemoglobin-related disorder in subject in need comprising a step of i) performing the in vitro method for monitoring the interruption of the treatment of haemoglobin-related disorder according to the invention and ii) adapting the treatment of haemoglobin-related disorder if it is concluded that said treatment exhibit not effect anymore.

According to the invention, adapting the treatment of haemoglobin-related disorders refers to adjust/increase the dosage of the compounds used to treat haemoglobin-related disorders and/or use an another compounds or therapy to treat haemoglobin-related disorders.

In other words, the invention refer to a treating haemoglobin-related disorder or monitoring the treatment of haemoglobin-related disorder in subject in need comprising a step of i) determining the concentration of AHSP in a sample obtained from the subject before or at the beginning of said treatment, ii) determining the concentration of AHSP in a sample obtained from the same subject at a second specific time of the treatment, iii) comparing the concentration of AHSP determined at step ii) with the concentration of AHSP determined at step i), and iv) continuing the treatment of haemoglobin-related disorder when the concentration of AHSP determined in step ii) is lower than the concentration of AHSP determined in step i) or adapting the treatment of haemoglobin-related disorder when the concentration of AHSP determined in step ii) is remained stable or higher than the concentration of AHSP determined in step i).

In another aspect, the invention refers to a method for treating haemoglobin-related disorder or monitoring the treatment of haemoglobin-related disorder in subject in need comprising a step of i) performing the in vitro method for diagnosing or prognosing haemoglobin-related disorder according to the invention and ii) administrating to said patient a therapeutically effective amount of a treatment of haemoglobin-related disorder if it is concluded that said subject has haemoglobin-related disorder.

In other words, the invention refers to a method for treating haemoglobin-related disorder or monitoring the treatment of haemoglobin-related disorder in subject in need comprising a step of i) determining the concentration of alpha-haemoglobin stabilising protein (AHSP) in a sample obtained from the subject, ii) comparing the concentration of AHSP determined at step i) with a reference value, and iii) administrating to said patient a therapeutically effective amount of a treatment of haemoglobin-related disorder when the concentration of AHSP determined at step i) are higher than the reference value.

As used herein, the term “treatment” or “treat” refer to curative or disease modifying treatment, to cure, delay the onset of, reduce the severity of, or ameliorate one or more symptoms of the haemoglobin-related disorder, or in order to prolong the survival of a subject beyond that expected in the absence of such treatment.

A “therapeutically effective amount” is intended for a minimal amount of active agent which is necessary to impart therapeutic benefit to a subject. For example, a “therapeutically effective amount” to a subject is such an amount which induces, ameliorates or otherwise causes an improvement in the pathological symptoms, disease progression or physiological conditions associated with or resistance to succumbing to a disorder. It will be understood that the total daily usage of the compounds used to treat haemoglobin-related disorder will be decided by the attending physician within the scope of sound medical judgment.

In some embodiments, the term “treatment of haemoglobin-related disorder” refers to therapy, such as gene therapy, bone marrow transplantation (to correct deficiencies in the synthesis or structure of haemoglobin chain for example) or compounds used to treat haemoglobin-related disorder such as sickle cell disease and/or beta-thalassaemia. The compounds used to treat haemoglobin-related disorder such as sickle cell disease or beta-thalassaemia includes but are not limited to erythrocyte-modifying treatments such as hydroxycarbamide, decitabine, decitabine/tetrahydouridine, panobinostat, pomalidomide, luspatercept and vepoloxamer; L-glutamine; voxelotor; erythropoiesis stimulating agents (ESA) such as erythropoietin (EPO), epoetin alfa, darbepoetin alfa and crizanlizumab, cobalamin, γ chain synthesis stimulating agent, iron and iron chelation therapy such as deferoxamine, deferiprone and deferasirox.

In some embodiments, the treatment of haemoglobin-related disorder is hydroxycarbamide.

As used herein, the term “hydroxycarbamide”, or “HC”, also known as “hydroxyurea” has its general meaning in the art and refers to a compounds having the following formula CH₄N₂O₂ currently used to treat sickle-cell disease and/or beta-thalassaemia, chronic myelogenous leukaemia, or cervical cancer. Its CAS number is 127-07-1. HC has been shown to increase HbF levels, thereby decreasing the rate of HbS polymerisation. HC treatment is often the first choice in SCD adult patients with symptomatic disease, to avoid transfusion and the risks it entails, and in children, to prevent further complications of the disease. However, the response to HC is highly variable and differs from one patient to another.

### 5) Kit of the Invention

Another aspect of the invention is a kit for use in the method of the invention as described here above, said kit comprising:
a solid support,
a human recombinant AHSP
a capture anti-AHSP antibody,
a detecting anti-AHSP antibody, and
instructions for use.

In one embodiment, the capture anti-AHSP antibody is coated directly or indirectly to a solid support, said solid support comprising a protein binding surface such as high-binding well ELISA plates; nitrocellulose (e. g., in membrane or microtiter well form); polyvinylchloride (e. g., sheets or microtiter wells); polystyrene latex (e.g., beads or microtiter plates); polyvinylidine fluoride; diazotized paper; nylon membranes; activated beads, magnetically responsive beads, and the like.

The invention will be further illustrated by the following figures and examples. However, these examples and figures should not be interpreted in any way as limiting the scope of the present invention.

### FIGURES

FIG. 1: Comparison of AHSP concentration in RBC lysates between controls (n=10), SS patients without HC treatment (n=12) and SS patients with HC treatment (n=10). AHSP concentration is expressed in μg AHSP per mL of haemolysate. The data are represented as boxplots, with the median and interquartile range indicated. Each point is the mean of four different experiments. The minimum values are 0.42, 1.39 and 1.18 for controls, untreated SS patients and HC-treated SS patients, respectively. The maximum values are 1.12, 2.90 and 2.16 for controls, untreated SS patients and HC-treated SS patients, respectively. The three groups were compared in Kruskal-Wallis analyses followed by Dunn's tests.

FIG. 2: Relationship between AHSP concentration and A) α-Hb pool, B) reticulocyte number, C) haemoglobin level, D) haematocrit, E) MCHbF level, or F) DRBC percentage. Controls (white circle, n=10), SS patients without HC treatment (stripped circle, n=12) and SS patients with HC treatment (black circle, n=10).

FIG. 3: Comparison of AHSP concentration in RBC lysates between β-thal patients (n=9) and controls (n=7).

This study concerns 9 β-thal patients (2 males and 7 females; mean age, 48.6±14.3 years) including 4 patients with an α triplication (ααα^{anti3.7}/αα) and 1 patient for which the α-globin genotype was not known. Three patients were treated by hydroxycarbamide and/or erythropoietin. The free AHSP concentration were compared to those observed in the control group (n=7) (4 males and 3 females: mean age, 47.4±13.4 years) using the Mann Whitney test. The AHSP concentration was measured by sandwich ELISA after a dilution of 1000-fold for the control haemolysates and 10,000-fold for the β-thal haemolysates. Taking into account the dilution factor, the SP was expressed in μg per mL of haemolysate. The data are represented as boxplots, with the median and interquartile range indicated. Each point is the mean of four different experiments and three different experiments for control group and β-thal group, respectively. The mean values are 1.22 and 18.36 μg/ml for controls and β-thal patients, respectively. The minimum values are 0.87, and 8.78 μg/ml for controls and β-thal patients, respectively. The maximum values are 1.48, and 25.70 μg/ml for controls, and β-thal patients, respectively.

FIG. 4: Comparison of AHSP concentration in RBC lysates between β-thal patients (n=9) and SS patients without HC treatment (n=12).

TABLE 1
Haematological data for the control group and for SS patients with and without HC treatment.
AHSP concentration is expressed in μg AHSP per mL of haemolysate. The data are represented as
boxplots, with the median and interquartile range indicated.

| | | | | Statistical difference |
|---|---|---|---|---|
| | | Untreated | Treated | between treated and |
| | Controls | SS patients | SS patients | untreated SS patients |
| | Mean ± SD | Mean ± SD | Mean ± SD | P value |
| Subjects | 10 | 12 | 10 | |
| Sex (F/M) | 6/4 | 6/6 | 7/3 | 0.3421 |
| Age (years) | 41.9 ± 12.8 | 39.8 ± 7.6 | 42.9 ± 10 | 0.4259 |
| WBC (×10⁹/L) | 6.47 ± 1.33 | 10.08 ± 2.49 | 6.88 ± 1.46 | 0.0037 |
| RBC (×10¹²/L) | 4.75 ± 0.29 | 2.75 ± 0.40 | 2.42 ± 0.45 | 0.0818 |
| Reticulocytes (×10⁹/L) | | 248.8 ± 87.25 | 114.7 ± 45.87 | 0.0003 |
| Reticulocytes (%) | | 9.16 ± 3.04 | 4.57 ± 1.79 | 0.0003 |
| Platelets (×10⁹/L) | 261 ± 22.98 | 487.6 ± 172.9 | 355.8 ± 125.5 | 0.0487 |
| RDW (%) | | 23.08 ± 4.27 | 17 ± 2.26 | 0.0007 |
| DRBCs (%) | | 11.92 ± 5.79 | 4.3 ± 2.54 | 0.0015 |
| MCV (fL) | 89.82 ± 3.07 | 88.08 ± 9.4 | 114.3 ± 13.43 | <0.0001 |
| Haematocrit (%) | 42.61 ± 1.74 | 24.33 ± 3.23 | 28.4 ± 4.74 | 0.0224 |
| Hb (g/dL) | 14.04 ± 0.79 | 8.23 ± 1.11 | 9.9 ± 1.58 | 0.0084 |
| MCHC (g/dL) | 32.95 ± 1.01 | 34.42 ± 0.90 | 34.8 ± 0.79 | 0.3928 |
| MCH (pg) | 29.57 ± 1.2 | 30.17 ± 3.51 | 39.7 ± 4.76 | 0.0001 |
| HbA₂ (%) | | 2.71 ± 0.40 | 2.04 ± 0.53 | 0.004 |
| HbF (%) | | 8.97 ± 6.25 | 21.51 ± 5.36 | 0.0003 |
| MCHbF (pg) | | 2.86 ± 2.33 | 8.69 ± 2.81 | 0.0002 |
| HbS (%) | | 88.33 ± 5.92 | 76.45 ± 4.9 | 0.0002 |
| MCHbS (pg) | | 26.50 ± 2.04 | 30.22 ± 2.89 | 0.0044 |
| Total bilirubin | | 43.5 ± 18.1 | 28.5 ± 14.18 | 0.0564 |
| (μmoL/L) | | | | |
| LDH (U/L) | | 445.4 ± 135.9 | 403.2 ± 68.09*# | 0.8078 |
| Ferritin (μg/L) | | 325.4 ± 318.2 | 333.2 ± 206.8 | 0.4965 |
| α-Hb pool (ppm) | 88 ± 17 | 232 ± 53 | 162 ± 31 | 0.0009 |

Abbreviations: F, female; M, male; WBC, white blood cell; RBC, red blood cell; RDW, red blood cell distribution width; DRBCs, dense dehydrated RBCs defined as having a density > 1.110 g/mL in RBC density distributions; MCV, mean corpuscular volume; MCHC, mean corpuscular haemoglobin concentration, MCH, mean corpuscular haemoglobin; MCHbF, mean corpuscular HbF, MCHbF = % HbF × MCH; MCHbS, mean corpuscular HbS, MCHbS = % HbS × MCH; LDH, lactate dehydrogenase.
The results are reported as the mean ± standard deviation (SD).
The significance of differences between SS patients with and without HC treatment was assessed in Mann-Whitney tests, with the exception of the variable “sex”, for which a chi-squared test was used.
#n = 9.
The α-Hb pool is expressed in ppm, equivalent to ng of free α-Hb per mg total (α ± non-α) subunits per mL of lysed RBCs.
AHSP concentration was measured by ELISA and is expressed as μg AHSP per mL of lysed RBCs.

TABLE 2
AHSP concentration in controls, and
in untreated and treated SS patients.

| | | |
|---|---|---|
| Group | Measured [AHSP] (μg/mL)^{a} | CV(%)^{b} |

Controls

| | | |
|---|---|---|
| T1 | 0.92 ± 0.20 | 22 |
| T2 | 0.96 ± 0.04 | 4 |
| T3 | 0.85 ± 0.15 | 18 |
| T4 | 1.12 ± 0.21 | 19 |
| T5 | 0.42 ± 0.07 | 16 |
| T6 | 1.03 ± 0.23 | 23 |
| T7 | 0.77 ± 0.04 | 5 |
| T8 | 0.58 ± 0.08 | 14 |
| T9 | 0.91 ± 0.19 | 21 |
| T10 | 0.61 ± 0.11 | 18 |
| Mean ± SD | 0.82 ± 0.22 | 16 ± 6.7 |

Untreated Patients

| | | |
|---|---|---|
| P1 | 2.30 ± 0.25 | 11 |
| P2 | 2.87 ± 0.40 | 14 |
| P3 | 1.44 ± 0.21 | 14 |
| P4 | 2.12 ± 0.68 | 32 |
| P5 | 1.39 ± 0.12 | 9 |
| P6 | 2.90 ± 0.46 | 16 |
| P7 | 1.79 ± 0.28 | 15 |
| P8 | 1.93 ± 0.195 | 10 |
| P9 | 2.16 ± 0.34 | 16 |
| P10 | 2.85 ± 0.30 | 10 |
| P11 | 2.48 ± 0.33 | 13 |
| P12 | 2.48 ± 0.41 | 16 |
| Mean ± SD | 2.23 ± 0.52 | 15 ± 6.0 |

Treated Patients

| | | |
|---|---|---|
| P13 | 1.37 ± 0.17 | 12 |
| P14 | 2.16 ± 0.29 | 13 |
| P15 | 1.99 ± 0.26 | 13 |
| P16 | 1.32 ± 0.13 | 10 |
| P17 | 1.18 ± 0.21 | 18 |
| P18 | 1.29 ± 0.23 | 18 |
| P19 | 1.19 ± 0.28 | 23 |
| P20 | 1.34 ± 0.15 | 11 |
| P21 | 1.44 ± 0.17 | 12 |
| P22 | 1.69 ± 0.15 | 9 |
| Mean ± SD | 1.50 ± 0.34 | 14 ± 4.4 |

^{a}The values are an average of 3 or 4 independent measurements of biological AHSP in haemolysates of control group (n = 10), untreated SS group (n = 12) and treated SS group (n = 10).
^{b}The inter-day precision was assessed by thecoefficient of variation (CV) determined by the formula: [SD of the mean/the mean value × 100].
Data are expressed as mean ± SD (standard
deviation).

TABLE 3
Correlation of AHSP concentration with haematological variables in controls, in SS patients
with and without HC treatment, and all subjects in pooled into a single group.

| | | | Untreated patients | | Treated patients | | All subjects | | |
|---|---|---|---|---|---|---|---|---|---|
| | Controls n = 10 | | n = 12 | | n = 10 | | (Controls and SS patients) | | |
| | r/rho* | P values | r/rho* | P values | r/rho* | P values | n | r/rho* | P values |
| Sex | | 0.3938** | | 0.2623** | | 0.7324** | | | 0.3472** |
| Age (years) | 0.3091* | 0.3869 | 0.2160 | 0.5000 | −0.2918* | 0.4112 | 32 | −0.0773* | 0.6740 |
| WBC (×10⁹/L) | 0.3333* | 0.3487 | −0.0301 | 0.926 | 0.6069 | 0.0628 | 32 | 0.6268* | 0.0001 |
| RBC (×10¹²/L) | −0.1394* | 0.7072 | 0.2998* | 0.3415 | 0.1287 | 0.7230 | 32 | −0.5879 | 0.0004 |
| Reticulocytes (×10⁹/L) | | | 0.6726 | 0.0166 | 0.1307 | 0.7189 | 22 | 0.7532 | <0.0001 |
| Reticulocytes (%) | | | 0.5601 | 0.0582 | 0.2310* | 0.5190 | 22 | 0.6975 | 0.0003 |
| Platelets (×10⁹/L) | 0.2066 | 0.5668 | −0.6949 | 0.0121 | −0.0916 | 0.8012 | 32 | 0.6075* | 0.0002 |
| RDW (%) | | | 0.0813 | 0.8016 | 0.2752* | 0.4369 | 22 | 0.5381* | 0.0098 |
| DRBCs (%) | | | −0.1158* | 0.7193 | 0.6236* | 0.0593 | 22 | 0.5452* | 0.0087 |
| MCV (fL) | 0.2409 | 0.5025 | 0.2884 | 0.3633 | 0.1273* | 0.7330 | 32 | 0.0488* | 0.7910 |
| Haematocrit (%) | 0.1399 | 0.6999 | 0.3215* | 0.3062 | 0.1116 | 0.7588 | 32 | −0.6990 | <0.0001 |
| Hb (g/dL) | 0.2851 | 0.4246 | 0.4316* | 0.1618 | 0.0962 | 0.7914 | 32 | −0.6869 | <0.0001 |
| MCHC (g/dL) | 0.3099 | 0.3836 | 0.0267 | 0.9336 | 0.0389* | 0.9244 | 32 | 0.4407 | 0.0116 |
| MCH (pg) | 0.4422 | 0.2006 | 0.2603 | 0.4139 | 0.0426 | 0.9069 | 32 | 0.0877* | 0.6332 |
| HbA₂ (%) | | | 0.1095* | 0.7342 | −0.3091 | 0.3848 | 22 | 0.4011* | 0.0643 |
| HbF (%) | | | 0.1636 | 0.6115 | 0.1218 | 0.7375 | 22 | −0.4500* | 0.0356 |
| MCHbF (pg) | | | 0.2308 | 0.4705 | −0.1152* | 0.7589 | 22 | −0.4692* | 0.0276 |
| HbS (%) | | | −0.1754 | 0.5856 | −0.1001 | 0.7833 | 22 | 0.4485* | 0.0363 |
| MCHbS (pg) | | | 0.1741 | 0.5884 | −0.0424* | 0.9184 | 22 | −0.4331* | 0.0441 |
| Total bilirubin (μmoL/L) | | | −0.0313 | 0.9230 | −0.4377* | 0.2069 | 22 | 0.1820 | 0.4175 |
| LDH (U/L) | | | −0.3147* | 0.3194 | −0.2030 | 0.6004 | 21 | −0.1247* | 0.5903 |
| Ferritin (μg/L) | | | −0.1903 | 0.5535 | −0.1585 | 0.6619 | 22 | −0.2309* | 0.3011 |
| α-Hb pool (ppm) | 0.3889 | 0.2667 | 0.6102 | 0.0351 | 0.6667 | 0.0352 | 32 | 0.8874 | <0.0001 |

The relationship between AHSP concentration and the various variables considered was analysed by calculating Pearson and Spearman* correlation coefficients, except for the variable “sex”, for which a Mann-Whitney test was used**. Abbreviations: F, female; M, male; WBC, white blood cell; RBC, red blood cell; RDW, red blood cell distribution width; DRBCs, dense dehydrated RBCs defined as having a density >1.110 g/mL in RBC density distributions; MCV, mean corpuscular volume; MCHC, mean corpuscular haemoglobin concentration. MCH, mean corpuscular haemoglobin; MCHbF, mean corpuscular HbF. MCHbF = % HbF × MCH; MCHbS, mean corpuscular HbS. MCHbS = % HbS × MCH; LDH. lactate dehydrogenase. The α-Hb pool is expressed in ppm, equivalent to ng of free α-Hb per mg total (α + non-α) subunits per mL of lysed RBCs

TABLE 4
Genotype and AHSP Concentration in β-thal patients.

| | | β mutation, | Associated | [AHSP] | CV |
|---|---|---|---|---|---|
| Patients | β phenotype | HGVS name | α mutation | (μg/mL)^{a} | (%) |
| 1*§ | β^{E}/β⁰ | HBB: c.79G > A/HBB: c.67G > T | | 25.63 ± 1.75 | 7 |
| 2§ | β⁰/β^{A} | HBB: c.92 + 1G > A/HR | ααα^{anti3-7}/αα | 8.78 ± 0.92 | 10 |
| 3 | β⁺/β⁺ | HBB: c.92 + 6T > C/HO | | 25.70 ± 3.41 | 13 |
| 4 | β⁰/β^{A} | HBB: c.382C > T/HR | | 15.99 ± 2.16 | 14 |
| 5 | β^{E}/β⁺ | HBB: c.79G > A/HBB: c.316-197C > T | | 21.70 ± 0.84 | 4 |
| 6 | β⁰/β^{A} | HBB: c.17_18delCT/HR | ααα^{anti3-7}/αα | 13.24 ± 1.58 | 12 |
| 7 | β⁰/β^{A} | HBB: c.118C > T/HR | ααα^{anti3-7}/αα | 23.53 ± 2.82 | 12 |
| 8* | β⁺/β^{A} | HBB IVS-I-5 (G > C) HR | ααα^{anti3-7}/αα | 12.78 ± 2.41 | 19 |
| 9 | β^{E}/β⁺ | HBB: c.79G > A/HBB: −28(A > G) | nd | 17.91 ± 4.11 | 23 |

HGVS, Human Genome Variation Society;
HO, homozygous;
HR, heterozygous;
*patients treated by hydroxycabamide
§patient treated by erythropoietin
^{a}The values are an average of 3 or 4 independent measurements of biological AHSP in haemolysates of β-thal group (n = 9).
Data are expressed as mean ± SD (standard deviation).
The inter-day precision was assessed by the coefficient of variation (CV) determined by the formula: [SD of the mean/the mean value × 100].

### Example 1

### Material & Methods

### Patients

The study was conducted in 12 SS patients not treated with HC, and 10 SS patients with stable HC treatment for at least one year, followed at the French Sickle Cell Referral Centre at Mondor Hospital in Creteil, France. Ten healthy adult volunteers without Hb abnormalities recruited by the French Blood Establishment (EFS) as blood donors were used as controls. All subjects were at least 18 years old. Informed consent was obtained from all subjects in accordance with the Declaration of Helsinki, after the project had been approved by the institutional review board. Data collection and sample storage were pseudo-anonymised.

SS patients were considered eligible for the study if they have four α-globin genes and were in a clinical steady state. Patients were considered to be in a steady state at visits attended more than 8 to 10 weeks after, or two weeks before any vaso-occlusive crisis or other clinical event resulting in hospitalisation. The exclusion criteria for all SS subjects were a transfusion within the last three months, active chronic viral disease (hepatitis B, C, HIV), current infection or a known inflammatory disease.

### Routine Clinical and Laboratory Data

Whole peripheral blood was collected from SS patients and controls into tubes containing EDTA as anticoagulant. Blood was collected from all patients for haematological and biological analyses. For all SS patients, Hb phenotyping was performed by Variant II cation exchange chromatography on an automatic analyser (BioRad, Hercules, CA, USA), and RBC density distributions were evaluated to determine the percentage of dense dehydrated RBCs (% DRBCs), defined as having a density >1.110 g/mL.²²

### Sample Preparation for Haemolysate Analysis

We centrifuged 4 mL of whole blood from each subject for 5 min at 4° C., with blood samples processed within 2 h of collection. The plasma was gently removed, leaving 3 mm above the RBCs to retain the reticulocytes; the RBCs were washed with 0.15 M NaCl and then frozen at −80° C. They were thawed on ice and lysed with four volumes of cold distilled water, and incubated for 30 min at +4° C., before centrifugation at 16,000×g for 30 min at +4° C. The RBC lysate (haemolysate) was recovered as the supernatant and stored at −80° C.

### Protein Preparations

Recombinant human AHSP was produced as a fusion protein with glutathione S-transferase (GST-AHSP) in E. coli BL21 (DE3) cells containing the pGEX6P2-AHSP expression plasmid, and was purified after cleavage of the GST moiety, as previously described.¹⁹ The concentration of recombinant AHSP was estimated with a coefficient of extinction of 11.46 mM⁻¹ cm⁻¹ at 280 nm. The recombinant AHSP was stored at −80° C. with phosphate-buffered saline (DPBS; Dulbecco's phosphate-buffered saline Gibco® Thermo Fisher Scientific, Waltham, MA, USA) containing with 10% glycerol.

Native α-Hb and β-Hb were prepared from normal adult human Hb, as previously described.¹⁹

### In Vitro α-Hb Pool Measurement in Haemolysate

The α-Hb pool was assessed for the RBC lysates of all subjects (SS patients and controls), by capturing α-Hb subunits on GST-AHSP bound to an affinity chromatography support, as previously described.¹⁵

### Western Blotting

The specificity of capture antibody was validated by western blotting. Purified recombinant AHSP, native α-Hb and β-Hb were analysed by sodium dodecyl sulphate-polyacrylamide gel electrophoresis (SDS-PAGE) (4-12% Bis Tris Bolt Gel, Thermo Fisher Scientific) and transferred to hydrophobic polyvinylidene difluoride (PVDF) membranes (Bio-Rad) with a semi-dry blotting system (Trans-Blot® Turbo™ transfer system, Bio-Rad). The membranes were blocked by incubation with 2% bovine serum albumin (BSA, A7030, Sigma Aldrich, St Louis, MO, USA) in Tris-buffered saline-Tween (TBS-T; 10 mM Tris-HCl, 150 mM NaCl, 0.05% Tween 20), and incubated for 2 h at room temperature with a rabbit monoclonal anti-Ahsp antibody developed against a synthetic peptide corresponding to the 50 amino acids at the C-terminus of human AHSP (EPR12319, Abcam, Cambridge, UK) diluted 1:2500 in TBS-T. The membrane was washed in TBS-T, and incubated for 2 h at room temperature with an alkaline phosphatase-conjugated anti-rabbit IgG secondary antibody (A3687, Sigma-Aldrich) diluted 1:7000 in TBS-T. The protein bands were detected by incubation with an alkaline phosphatase substrate, 5-bromo-4-chloro-3-indolyl phosphate p-nitro-blue tetrazolium chloride (BCIP/NBT) (SigmaFast™ BCIP®/NBT Tablets, Merck, Darmstadt, Germany) for an appropriate time, followed by imaging on a Gel Doc® XR⁺ (Bio-Rad).

### Sandwich ELISA

We used high-binding 96-well ELISA plates (COSTAR High Binding ELISA Plates, Corning Incorporated, Corning, NY, USA). Following each step, up to 3,3′,5,5′-tetramethylbenzidine (TMB) addition, the wells were washed three times with 300 μL DPBS-0.05% Tween-20 (DPBS-TWEEN® Tablets, Calbiochem, Millipore Corporation, Burlington, MA, USA). The wells were coated with 100 μL/well anti-Ahsp antibody (EPR12319, diluted 1:1000 in DPBS) as the capture antibody. After 2 h at 37° C., the wells were washed and blocked by incubation for 2 h at 37° C. with 300 μL blocking buffer (0.15% BSA in DPBS). The wells were washed and left to dry in air, covered with SealPlate and stored at 4° C.

Serial dilutions of recombinant AHSP stored in −80° C. were prepared (0.395 to 3 ng/mL) in blocking buffer and added to the plate in duplicate in each assay, for construction of the calibration curve. Diluted recombinant AHSP were added to the coated wells (100 μL/well) and the plates were incubated for 2 h at 37° C. The plates were washed and 100 μL of biotin-conjugated rabbit polyclonal anti-human recombinant Ahsp antibody (Met1-Ser102) (ABIN2615200, Antibodies-online Gmb, Aachen, Germany, diluted 1:255 in blocking buffer), used as the detecting antibody, were added to each well, and the plates were incubated for 1 h at 37° C. The plates were washed five times and the samples were then incubated with 100 μL/well streptavidin-HRP (DY998, R&D Systems, Minneapolis, MN, USA, diluted 1:100 in blocking buffer) for 20 min at 37° C. in the dark. Next steps, washes and incubations, were performed in the dark. The reaction was developed by adding 50 μL/well TMB as a substrate (1-Step™ Ultra TMB-ELISA, Thermo Fisher Scientific). The plates were incubated for 5-10 min, and the reaction was then stopped by adding 50 μL/well 1 N sulfuric acid. Absorbance was measured immediately, at 450 nm (A₄₅₀), with a microplate reader (Eon™, BioTek Instruments Inc, Winooski, Vermont, USA).

A standard curve was generated with known concentrations and absorbances of the recombinant human AHSP, with the EON microplate reader and Gen5 software.

The intra-assay variability was assessed by calculating coefficients of variation (CVs) for two series of recombinant AHSP determinations used for calibration and deposited in duplicate on the same plate on the same day. The inter-day assay variability was calculated for four determinations (4 days) of recombinant AHSP deposited in duplicates.

We also determined the capacity of this ELISA to detect the human AHSP/α-Hb complex. The recombinant AHSP/α-Hb complex was prepared by adding 32 μM purified native α-Hb¹⁹ to recombinant AHSP in the same range of concentrations used to generate the standard curve. The AHSP/α-Hb complex was loaded onto the plates instead of recombinant AHSP alone, and the experiment was then performed as described above.

Determination ofAHSP Concentration in Haemolysates by Sandwich ELISA

The haemolysates were diluted 1:1000 in blocking buffer and added (100 μL/well) to the plate coated with the capture antibody. The plates were incubated for 2 h at 37° C., washed and 100 μL of the detecting antibody were added to each well, and then the plates were incubated for 1 h at 37° C. The further procedure is the same as for the AHSP range described above. The AHSP concentrations of the diluted haemolysates were determined by comparison to the standard curve expressed as ng per mL by the microplate reader. All the concentrations of AHSP obtained are the means of four ELISAs. Finally, taking into account the 1000-fold dilution of haemolysates, the final concentrations are reported in μg AHSP per mL of haemolysate.

The inter-subject variabilities were determined by calculating the CVs for four independent measurements of biological AHSP in each haemolysate.

### Statistical Analysis

Quantitative variables are expressed as the arithmetic mean±standard deviation (SD). The comparisons were performed with Mann-Whitney tests for two groups or Kruskal-Wallis tests with Dunn's tests for multiple comparisons for multiple groups. Chi-squared tests were used for comparisons of the variable “sex” between groups. Correlations were analysed by calculating Spearman's rho and Pearson's correlation coefficients. The interaction of all variables with AHSP concentration was assessed by stepwise multivariate analysis. If a significant correlation was found between variables, only one of the variables concerned was included in the model, to prevent multicollinearity. All data processing and statistical analyses were performed with Prism 6.00 software (GraphPad Inc, La Jolla CA, USA) and Stata/Se version 12.0 software (StataCorp LP, College Station, TX, USA). P values <0.05 in two-tailed tests were considered significant.

### Results

### Characteristics of the Populations Studied

The adult population studied was described in a previous article on evaluation of the soluble α-Hb pool in RBCs from controls (n=10), SS patients without HC treatment (n=12) and SS patients with HC treatment (n=10).¹⁷ The haematological and clinical data for the two SS patient groups and the controls are summarised in Table 1. No differences in age or sex were found between the three groups. As expected, HC treatment was associated with a significant increase in % HbF and a significant decrease in DRBC percentage (8.97 vs. 21.51%, P=0.0003, 11.92 vs. 4.3%, P=0.0015, respectively).^{12,22}

### ELISA Validation

For ELISA optimisation, the concentrations of anti-AHSP (capture antibody) and biotin-conjugated Anti-AHSP antibodies (detection antibody) used were selected so as to obtain the signal/noise ratio. The specificity of the capture antibody was assessed by western blotting with purified recombinant AHSP and purified native α-Hb and β-Hb as negative controls (data not shown). The results obtained confirmed that this antibody was highly specific, recognising only the protein band corresponding to recombinant human AHSP but not the bands corresponding to α-Hb or β-Hb. We also assessed the specificity of interactions between recombinant human AHSP and the capture and detection antibodies on ELISA plates, by evaluating the signal obtained in the absence of AHSP. In the absence of recombinant AHSP, no signal was detected, giving a background value of 0 (data not shown). Thus, any signal detected after the addition of the substrate was due to specific binding of the detection antibody to the AHSP on the plate.

The ELISA was calibrated with recombinant human AHSP by analysing calibration standards, with a first dilution of 1:5000 in blocking buffer, followed by successive dilutions in blocking buffer to achieve a range of concentrations from 0.2 to 5 ng/mL. The final concentrations of AHSP were reported by the plate reader in ng/mL. A linear dose response was observed at relatively low concentrations, and the working range of this ELISA method was 0.395-3 ng/mL (data not shown).

The intra-assay variability was assessed by the CVs of two series of recombinant AHSP determinations (data not shown). In one of the two sets of values obtained on the same day, the CV ranged from 1.73 to 13.24% and there were four CV values below 10%. In the other set, the CV ranged from 1.95 to 21.46% with five values below 15%. Repeatability was, therefore, considered acceptable. We also assessed the inter-day assay variability of our method (data not shown). All CVs were below 8%. The recovery range of intra- or inter-assay were from 97 to 107%. The repeatability and reproducibility of the assay were therefore considered to be good, indicating that this assay is suitable for the quantitative detection of AHSP in haemolysates.

We also evaluated the capacity of the ELISA to detect the human biological AHSP/α-Hb complex, which may be present in haemolysates. Instead of loading the wells with recombinant AHSP alone, we loaded them with recombinant AHSP/α-Hb complex, and the ELISA plates were then processed in the same way as for AHSP alone. Contrary to what was observed for AHSP alone, no absorbance was detected at 450 nm, indicating that our method was unable to recognise this complex. In our experiments with haemolysates, we therefore measured exclusively the free biological soluble AHSP fraction and did not detect the fraction of AHSP complexed to α-Hb in RBCs.

### Biological AHSP Concentration in the Haemolysates of all Subjects

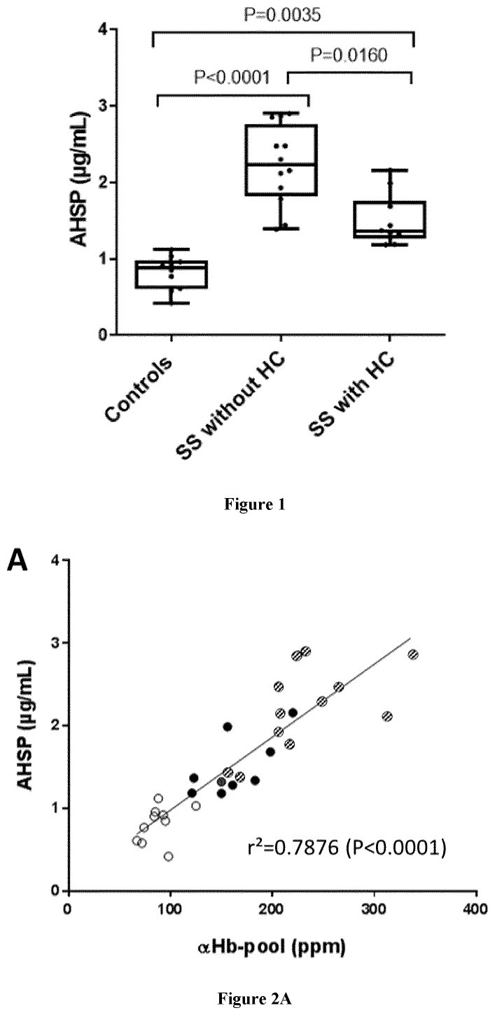

The final concentrations of AHSP in the three groups studied are reported in μg/mL, taking into account the dilution factor of 1000 of the haemolysate (FIG. 1 and Table 2). In the control group (n=10), mean AHSP concentration was 0.82±0.22 μg/mL, and the values obtained ranged from 0.42 to 1.12 μg/mL (interquartile range (IQR)=0.60-0.98). The CVs ranged from 4 to 23% and the mean CV was 16±6.7%. In the untreated SS patient group (n=12), AHSP concentrations were significantly higher than those in the control group (2.23±0.52 vs. 0.82±0.22 μg/mL P<0.0001), and ranged from 1.39 to 2.9 μg/mL (IQR=1.82-2.76). The CVs ranged from 9 to 32% and the mean CV values were 15±6.0%. Patients treated with HC had significantly lower AHSP concentrations than untreated SS patients (1.50±0.34 vs. 2.23±0.52 μg/mL, respectively, P=0.0160) with a lesser dispersion of AHSP concentrations (IQR=1.26-1.76). However, AHSP concentration remained significantly higher than that in controls (P=0.0035). The values in this patient group ranged from 1.18 to 2.16 μg/mL and the CV ranged from 9 to 23% and the mean CV was 14±4.4%.

### Correlations Between AHSP Concentration and Clinical and Haematological Data

We analysed the correlations between AHSP concentration and nine clinical and haematological characteristics for controls and 21 clinical and haematological characteristics for SS patients with and without HC treatment, in univariate analyses (Table 3). In this table, we indicated the strongest correlations obtained either with Pearson's correlation coefficient (r) or with Spearman's correlation coefficient (rho). No significant differences in AHSP concentration according to sex or age were observed in the patients or controls. In the control group, all correlations of AHSP concentration with the nine variables considered had P-values greater than 0.2. For the group of untreated SS patients, P-values for the correlation with AHSP concentration were below 0.05 for three variables: reticulocytes (r=0.6726, P=0.0166), platelets (r=−0.6949, P=0.0121) and α-Hb pool (r=0.6102, P=0.0351). For the treated SS patients, only α-Hb pool was significantly correlated with AHSP concentration (r=0.6667, P=0.0352).

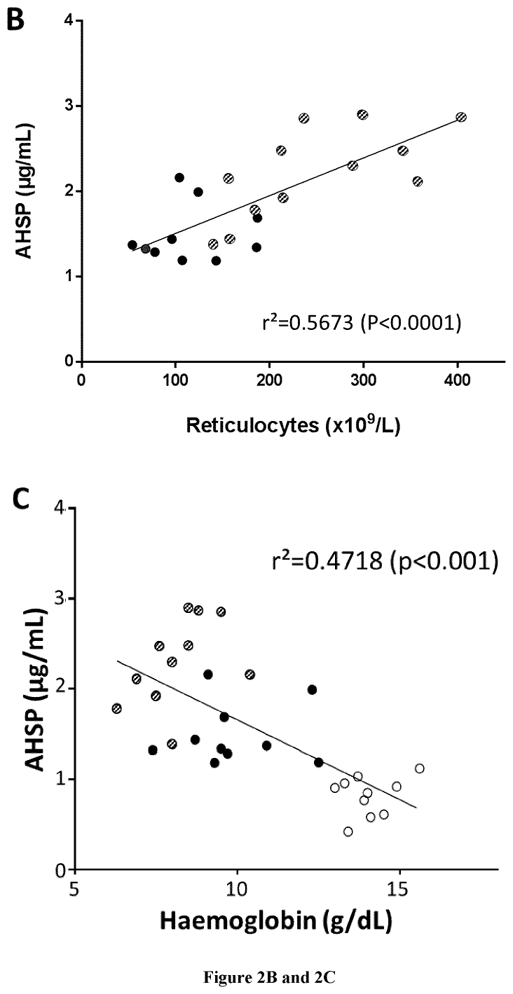

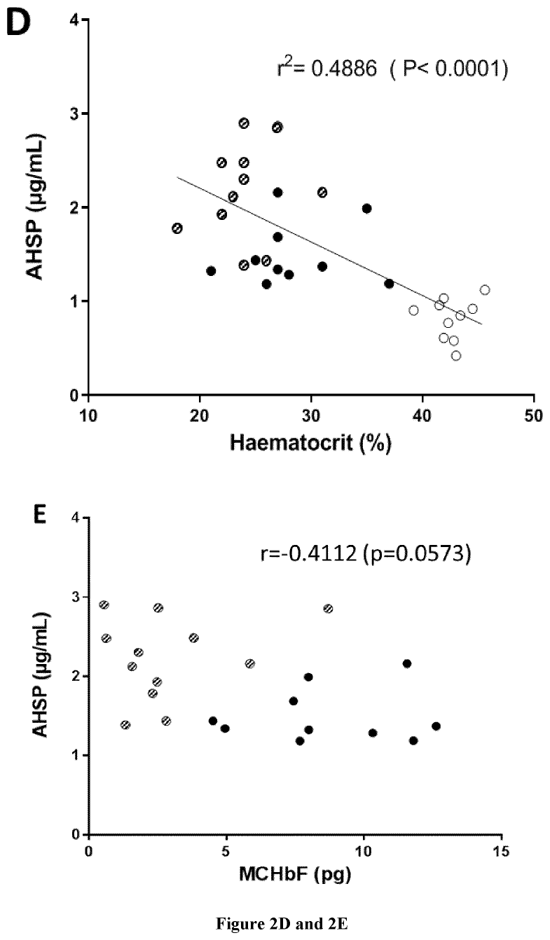

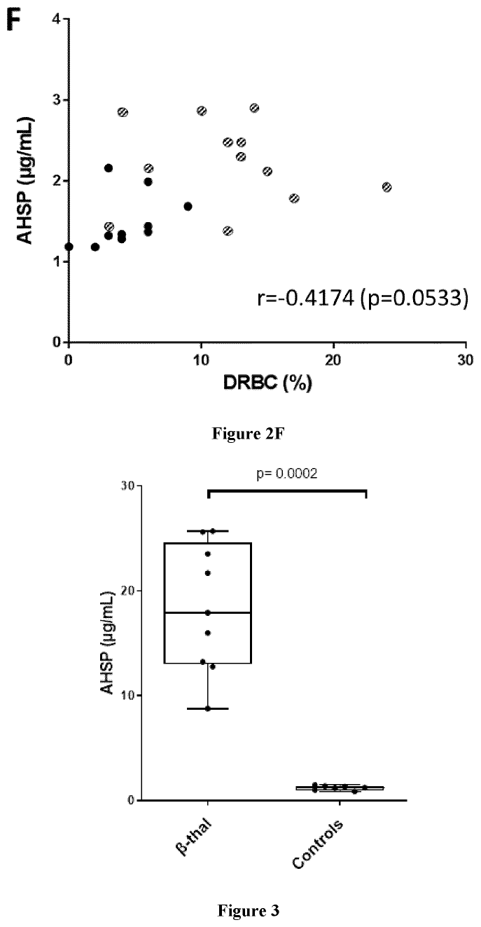

Given the small numbers of individuals in each group, we reanalysed the correlations between AHSP concentration and the various factors by considering the controls, treated and untreated SS patients as a single group (n=32) (Table 3). AHSP concentration was not correlated with sex or age. Four variables displayed significant linear regressions with AHSP concentration, with a correlation coefficient of about 0.7 (Table 3). α-Hb pool was the variable most strongly correlated with AHSP concentration (n=32, r=0.8874, P<0.0001) (FIG. 2A). The other three variables were reticulocytes (n=22, r=0.7532, P<0.0001), Hb (n=32, r=−0.6869, P<0.0001) and haematocrit (n=32, r=−0.6990, P<0.0001) (FIG. 2A-D). Two other variables sensitive to HC treatment presented weak correlations with AHSP: mean corpuscular HbF (MCHbF) and % DRBCs. For the relationship between AHSP concentration and MCHbF (n=22), the Spearman's coefficient (rho=−0.4692, P=0.0276) is greater than the Pearson's coefficient (r=−0.4112, P=0.0573) indicating a weak non-linear association (FIG. 2E). Such a weak non-linear association was also observed between AHSP concentration and % DRBCs (n=22; rho=0.5452, P=0.0087; r=0.4174, P=0.0533) (FIG. 2F). The interaction between AHSP concentration and these variables was independent of patient treatment or individual status. Multivariate analysis showed no significant associations with AHSP concentration for any of the variables other than α-Hb pool.

### DISCUSSION

AHSP plays an important role in the physiological process of erythropoiesis,^{1,18} and may also be a modulator of Hb disorders, such as β-thal.³⁻⁵ We recently showed that the free α-Hb pool of SCA patients is significantly higher than that of controls,¹⁶ and that this α-Hb pool decreases following HC treatment in SS patients.¹⁷ Certain cytosolic proteins of RBCs have been reported to be upregulated, like AHSP, in SCA.¹¹ We hypothesised that AHSP concentration increased in these patients to compensate for the relative excess of free α-globin chains. We developed a sandwich ELISA method to quantify biological AHSP using recombinant AHSP as standard. The high recovery rates (97 to 107%) and acceptable CV values (<22%) for intra- and inter-assay suggest that this ELISA has an acceptable performance and can be used as a good tool for quantification of AHSP in RBCs. Using a sandwich ELISA procedure, we quantified, for the first time, the concentration of AHSP in the RBCs of healthy subjects. We also demonstrated an upregulation of AHSP protein levels in the RBCs of SCA patients. However, this ELISA cannot detect AHSP complexed with the a subunit present in RBCs, indicating that the capture or detection antibodies recognise the same AHSP domain as the a subunit. This method can therefore be used exclusively for the quantification of free biological soluble AHSP, and not AHSP bound to a chains. This ELISA can detect as little as 0.4 ng human AHSP/mL.

Consistent with the findings for AHSP gene expression, AHSP protein levels were found to be unrelated to age or sex.^{4,5} The interindividual variability of AHSP concentration was high in control subjects (up to three-fold). This finding is consistent with the AHSP transcript levels in the circulating reticulocytes of healthy individuals.⁴ Mean AHSP concentration was 0.82 μg/mL haemolysate, corresponding to 1.8×10⁴ AHSP molecules per RBC. This is much lower than the at least at 10⁷ AHSP molecules per late erythroid precursor estimated by Kihm and collaborators.¹ AHSP expression gradually increases during erythroid development, peaking at the polychromatic and orthochromatic stages.¹⁸ Walczak et al. recently used imaging flow cytometry to study the development of erythroblasts from healthy donors. They found that, at late stages of maturation, the AHSP protein was present predominantly in the nucleus and was not longer detectable in the reticulocytes devoid of nuclei.²³ By contrast, Basu and coworkers showed, by proteomic techniques, that AHSP was present in erythrocytes purified from fresh blood from controls and patients with SCD.¹¹ Here, we also demonstrated the presence of AHSP molecules in RBCs from both controls and SCA patients.

AHSP concentrations in the RBCs of the SS patients without HC treatment were significantly higher (by a factor of 2.7) than those in controls. These results are consistent with those reported for normal subjects and Hb-depleted cytosol from SCD patients by Basu and coworkers.¹¹ HC treatment significantly decreased AHSP concentration in SS patients, potentially reflecting the magnitude of the change in erythropoiesis induced by HC. Indeed, using in vitro and in vivo derived human erythroblasts, the group of El Nemer recently published evidence an ineffective erythropoiesis in SCD^{24,25}. The increase in Hb F level in cultures treated with pomalidomide was shown to have beneficial effect on anaemia not only due to the longer life span of RBCs but also due to lower levels of ineffective erythropoiesis.²⁴ They speculate that treatments, such as HC, targeting HbS expression or polymerisation would inhibited apoptosis and increase reticulocyte count.²⁵ If HC treatment inhibited apoptosis and corrected ineffective erythropoiesis, this can explain the decrease of α-Hb pool and AHSP concentration in RBCs as observed in our study.

The few studies to date investigating associations between AHSP levels and haematological variables were performed on thalassaemia patients, by RT-qPCR.^{5,6} Given the small number of patients in each group in our study, due to the fact that the study was initially designed to investigate the effect of HC on the α-Hb pool,¹⁷ we reanalysed our data with the three groups of subjects pooled into a single group. Univariate analysis revealed a strong correlation between AHSP concentration and the α-Hb pool. We showed, in our previous study on the same SS patients, that the α-Hb pool was strongly correlated with the number of reticulocytes.¹⁷ The correlation between AHSP concentration and reticulocyte number observed here was, therefore, expected. Multivariate analysis revealed no interaction between AHSP and these variables other than the association with α-Hb pool. The strong correlation between AHSP concentration and α-Hb pool is not surprising because the results obtained here for protein levels are consistent with those previously obtained for gene expression. Indeed, AHSP gene expression has been shown to increase steadily following expression of the α-globin gene during the maturation of normal RBC precursors²² and a positive correlation has been reported between the levels of excess α-globin [α−(β+γ)] mRNA and the level of AHSP mRNA.²⁶

It has also been shown that AHSP expression in the reticulocytes of β-thal patients is significantly correlated with excess α-globin expression.⁵ Finally, we found no correlation between AHSP concentration and ferritin levels, despite the demonstration that AHSP gene expression is regulated by an iron-responsive element;²⁷ all our patients had normal ferritin concentrations. These results should be interpreted as a proof-of-concept, given the small number of patients studied, and larger populations of SS and thalassaemia patients will need to be studied to determine the clinical utility of AHSP measurement determinations.

The correlations of certain haematological variables with AHSP mRNA or AHSP protein levels in erythrocytes suggest a possible regulation of translation. Our method for AHSP determination in erythrocytes could be used to monitor, at protein level, the results of the regulation of AHSP translation, and could provide a new approach for monitoring the pharmacological or genetic modification of AHSP expression, and the severity of haemoglobinopathies.

We previously showed that the α-Hb pool in the RBCs of untreated SS patients was higher than that in the RBCs of controls and that this pool was decreased by HC treatment. We show here, in the same group of patients, that AHSP concentration is also higher in the RBCs of untreated SS patients than in those of controls, and that this concentration is decreased by HC treatment and associated with the α-Hb pool. AHSP concentration may therefore be a better new RBC biomarker of SCA disease that is easier to measure than the α-Hb pool. It could be used as a surrogate marker for monitoring the response to erythrocyte-modifying treatments and for monitoring patient compliance with such treatments. As AHSP concentration is not correlated with bilirubin or LDH levels, it may be an independent marker of erythrocyte life patterns. The AHSP pool partly stabilises the excess α-Hb pool by binding to α-Hb monomers, keeping them in a stable, soluble form. The remaining excess α-Hb exceeding the capacity of AHSP is precipitated and degraded in the cell. The stimulation of AHSP synthesis may, therefore, be a useful approach for improving the phenotype of diseases involving the accumulation of excess α-Hb. In particular, it has been shown that, in β⁰-thalassaemic erythroblasts, gene expression remains unchanged during erythroid maturation, except for the AHSP gene, the expression of which increases significantly.²⁶ Excess α-globin may therefore drive the expression of AHSP in both normal and pathological erythroid cells, implying the existence of an unknown feedback mechanism by which erythroid cells modify AHSP levels.

The range of AHSP concentrations observed in patients is large. A genetic approach could therefore be considered, because polymorphisms have been reported to modify AHSP expression.⁴

We have shown, in β-thal intermedia patients, that the α-Hb pool reflects the combined effects of a set of genetic factors that alter the production of the free α-Hb chain.¹⁵ It would be interesting to determine AHSP protein levels in these patients to determine whether AHSP could, like α-Hb pool, be used as a new marker to guide the diagnosis of β-thal intermedia.

In conclusion, we have developed an ELISA method, which we used to quantify the AHSP protein in human RBCs for the first time. This method was successfully applied to RBCs from 10 healthy controls, 12 SS patients without HC treatment and 10 SS patients with HC treatment. We showed that AHSP was significantly more abundant in untreated SS patients than in controls. AHSP concentration decreased with HC treatment but remained significantly higher than that in controls. AHSP concentration was found to be strongly correlated with the soluble α-Hb pool, regardless of treatment. This ELISA could provide a method for monitoring the regulation of AHSP translation and will facilitate the study of AHSP in different pathological conditions, for evaluation of its potential for use as a biomarker in RBCs.

### Example 2

We recently developed an α-Hb pool measurement and demonstrated that the soluble α-Hb pool in RBC lysates from β-thal intermediate patients was highly significant increased compared to control subjects and was a new marker to characterize these patients and may be clinically useful for characterizing and monitoring the evolution of the disequilibrium of globin synthesis in response to treatments. We also showed the presence of an increased soluble α-Hb pool in the RBC lysates of sickle cell anemia (SCA) patients (SS homozygous patients) but to a lesser extent than in β-thal (WO/2010/122160).

Based on an enzyme immunoassay method (ELISA) we developed, we can precisely quantify the amount of AHSP protein in RBCs. We demonstrated a significantly increased AHSP concentration in RBCs of patients with SCA compared to control subjects. We also showed that this AHSP concentration was correlated with the α-Hb pool.

Accordingly to these data, we hypothesized that in β-thal patient's RBCs, the free AHSP concentration should also be highly increased such as the α-Hb pool and may be useful in predicting the severity of the disease.

We investigated 9 β-thal patients including 4 β-thal patients without α-thalassemia, 4 patients with an α triplication (ααα^{anti3.7}/αα). and 1 patient for which the α-globin genotype was not known (Table 6). Seven controls subjects with Hb A were also studied. The determination of AHSP concentration was achieved directly from the lysate of RBCs using the ELISA described in example 1. This ELISA was performed on the control haemolysates diluted 1000-fold and on the f-thal haemolysates diluted 10,000-fold. The final concentrations of AHSP were reported in g/ml, taking into account the dilution factor.

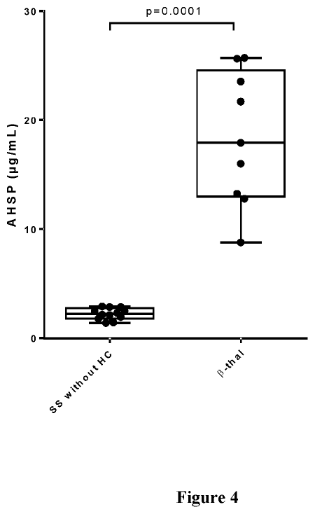

As expected and like the alpha-Hb pool, the mean of free AHSP concentration in the β-thal patients haemolysates was greatly increased [18.36±6.12 μg/ml] compared to both control subjects (FIG. 3) and SCA patients (FIG. 4, [2.23±0.52 μg/ml]).

### REFERENCES

Throughout this application, various references describe the state of the art to which this invention pertains. The disclosures of these references are hereby incorporated by reference into the present disclosure.
1. Kihm A J, Kong Y, Hong, W, et al. An abundant erythroid protein that stabilizes free α-haemoglobin. Nature. 2002; 417(6890):758-763.
2. Gell D, Kong Y, Eaton S A, et al. Biophysical characterization of the alpha-globin binding protein alpha-hemoglobin stabilizing protein. J Biol Chem. 2002; 277(43):40602-40609.
3. Kong Y, Zhou S, Kihm A J, et al. Loss of alpha-hemoglobin-stabilizing protein impairs erythropoiesis and exacerbates beta-thalassemia. J Clin Invest. 2004; 114(10):1457-1466.
4. Lai M I, Jiang J, Silver N, Best S, et al. Alpha-haemoglobin stabilising protein is a quantitative trait gene that modifies the phenotype of beta-thalassaemia. Br J Haematol. 2006; 133(6): 675-682.
5. Lim W F, Muniandi L, George E, et al. α-Haemoglobin stabilising protein expression is influenced by mean cell haemoglobin and HbF levels in HbE/β-thalassaemia individuals. Blood Cells Mol Dis. 2012; 48(1):17-21.
6. Mahmoud H M, Shoeib A A, Abd El Ghany S M, et al. Study of alpha hemoglobin stabilizing protein expression in patients with β thalassemia and sickle cell anemia and its impact on clinical severity. Blood Cells Mol Dis. 2015; 55(4):358-362.
7. dos Santos C O, Costa F F. AHSP and beta-thalassemia: a possible genetic modifier. Hematology. 2005, 10, 157-61.
8. Yu H, Pinkus J L and Pinkus G S. Alpha-hemoglobin-stabilizing protein: an effective marker for erythroid precursors in bone marrow biopsy specimens. Appl Immunohistochem Mol Morphol. 2016; 24(1):51-56.
9. Zhu G Z, Yang Y L, Zhang Y J, et al. High expression of AHSP, EPB42, GYPC and HEMGN predicts favorable prognosis in FLT3-ITD-negative acute myeloid leukemia. Cell Physiol Biochem. 2017; 42(5):1973-1984.
10. Surapolchai P, Chuansumrit A, Sirachainan N, et al. A molecular study on the role of alpha-hemoglobin-stabilizing protein in hemoglobin H disease. Ann Hematol. 2017; 96(6):1005-1014.
11. Basu A, Saha S, Karmakar S, et al. 2D DIGE based proteomics study of erythrocyte cytosol in sickle cell disease: altered proteostasis and oxidative stress. Proteomics. 2013; 13(21):3233-3242.
12. McGann P T, Ware R E. Hydroxyurea therapy for sickle cell anemia. Expert Opin Drug Saf 2015; 14(11):1749-1758.
13. Weatherall D J. Thalassaemia: the long road from bedside to genome. Nat Rev Genet. 2004 Aug; 5(8):625-31. doi: 10.1038/nrg1406. PMID: 15266345.
14. Vasseur C, Pissard S, Domingues-Hamdi E, et al. Evaluation of the free α-haemoglobin pool in red blood cells: a new test providing a scale of β-thalassaemia severity. Am J Hematol. 2011; 86(2):199-202.
15. Vasseur C, Domingues-Hamdi E, Ledudal K, et al. Red blood cells free α-haemoglobin pool: a biomarker to monitor the β-thalassemia intermedia variability. The ALPHAPOOL study. Br J Haematol. 2017; 179(1):142-153
16. Vasseur C, Domingues-Hamdi E, Pakdaman S, et al. Elevated soluble α-hemoglobin pool in sickle cell anemia. Am J Hematol 2017; 92(10):E593-E595.
17. Domingues-Hamdi E, Vasseur C, Pakdaman S, et al. Hydroxycarbamide decreases the free alpha-hemoglobin pool in red blood cells of adult patients with sickle cell anemia. Am J Hematol. 2020; 95(11), E302-E305.
18. dos Santos C O, Duarte A S, Saad S T, et al. Expression of alpha-hemoglobin stabilizing protein gene during human erythropoiesis. Exp Hematol. 2004; 32(2):157-162.
19. Brillet T, Baudin-Creuza V, Vasseur C, et al. Alpha-hemoglobin stabilizing protein (AHSP), a kinetic scheme of the action of a human mutant, AHSPV56G. J Biol Chem. 2010; 285(23):17986-17992.
20. Baudin-Creuza V, Vasseur-Gobillon C, Pato C, préhu C, Wajcman H, and Marden M. C. Transfer of Human α- to β-Hemoglobin via Its Chaperone Protein. J Biol Chem. 2004; 279(35):36530-3.
21. Giardine et al, Nucleic Acids Res. 2014 January; 42; and on https://globin.bx.psu.edu/hbvar/menu.html.
22. Bartolucci P, Brugnara C, Teixeira-Pinto A, et al. Erythrocyte density in sickle cell syndromes is associated with specific clinical manifestations and hemolysis. Blood. 2012; 120(15):3136-3141.
23. Walczak J, Camargo Johnson M D and Muthumalaiappan K. Stage-specific expression pattern of alpha-hemoglobin-stabilizing-protein (AHSP) portrayed in erythroblast chronology. Methods Protoc. 2020:3(3):E46.
24. El Hoss S, Cochet S, Godard A, Yan H, Dussiot M, Frati G, Boutonnat-Faucher B, Laurance S, Renaud O, Joseph L, Miccio A, Brousse V, Mohandas N, El Nemer W. Fetal hemoglobin rescues ineffective erythropoiesis in sickle cell disease. Haematologica. 2020. doi: 10.3324/haematol.2020.265462.
25. El Nemer W, Godard A, El Hoss S. Ineffective erythropoiesis in sickle cell disease: new insights and future implications. Curr Opin Hematol. 2021 May 1; 28(3):171-176. doi: 10.1097/MOH.0000000000000642.
26. Varricchio L, Fabucci M-E, Alfani E, Godbold J, and Migliaccio A-R. Compensated variability in the expression of globin-related genes in erythroblasts generated ex vivo from different donors. Transfusion. 2010; 50:672-684.
27. dos Santos C O, Dore L C, Valentine E, et al. An iron responsive element-like stem-loop regulates alpha-hemoglobin-stabilizing protein mRNA. J Biol Chem. 2008; 283(40):26956-64.

1. (canceled)

2. (canceled)

3. (canceled)

4. (canceled)

5. (canceled)

6. (canceled)

7. (canceled)

8. (canceled)

9. (canceled)

10. (canceled)

11. An in vitro method for determining the concentration of alpha-haemoglobin stabilising protein (AHSP) in a sample obtained from a subject, comprising the steps of i) contacting said sample with a first and a second antibody, each of which specifically binds to AHSP, ii) measuring the amount of the first and the second antibody bound to AHSP and iii) calculating the concentration of AHSP in the sample based on the amount of bound antibody.

12. The in vitro method according to claim 11, wherein the first antibody is a capture antibody that specifically binds to AHSP, and the second antibody that specifically binds to AHSP is a detecting antibody that is coupled to biotin.

13. The in vitro method according to claim 12, wherein the sample is first contacted with the capture antibody, and then with the detecting antibody.

14. The in vitro method according to claim 13, wherein a protein coupled with an enzyme is added to the sample after the detecting antibody.

15. (canceled)

16. A method for treating and monitoring a haemoglobin-related disorder in subject in need thereof comprising i) determining a first concentration of AHSP in a sample obtained from the subject at a first specific time, then
ii) treating the subject for the haemoglobin-related disorder, then
iii) determining that a second concentration of AHSP in a sample obtained from the subject at a second specific time is the same or higher than the first concentration,
and iv) administering to the subject a different and/or increased amount of a treatment for the haemoglobin-related disorder, wherein the treatment is selected from gene therapy, bone marrow transplantation, an erythrocyte-modifying compound, an erythropoiesis stimulating agent, iron and iron chelation therapy.

17. A method for treating and monitoring a haemoglobin-related disorder in subject in need thereof comprising i)
i) determining a first concentration of AHSP in a sample obtained from the subject at a first specific time, then
ii) treating the subject for the haemoglobin-related disorder, then
iii) determining that a second concentration of AHSP in a sample obtained from the subject at a second specific time is lower than the first concentration of AHSP,
and iv) continuing treatment of the haemoglobin-related disorder in the subject, wherein the treatment is selected from gene therapy, bone marrow transplantation, an erythrocyte-modifying compound, an erythropoiesis stimulating agent, iron and iron chelation therapy.

18. A method for diagnosing and treating a haemoglobin-related disorder in subject in need thereof comprising i) determining in a sample obtained from the subject a concentration of AHSP, and
ii) administering to a patient identified as having a concentration of AHSP higher than a corresponding reference value a therapeutically effective amount of a treatment for the haemoglobin-related disorder wherein the treatment is selected from gene therapy, bone marrow transplantation, an erythrocyte-modifying compound, an erythropoiesis stimulating agent, iron and iron chelation therapy.

19. The method according to claim 16, wherein the concentration of AHSP determined at the step i) is determined when the subject is in steady state.

20. The method according to claim 18, wherein the haemoglobin-related disorder is sickle cell disease or beta-thalassaemia.

21. The method according to claim 18, wherein the treatment of the haemoglobin-related disorder is hydroxycarbamide.

22. The method according to claim 18, wherein the concentration of AHSP is determined by enzyme-labeled and mediated immunoassays (ELISA).

23. The method of claim 22, wherein the ELISA is a sandwich ELISA.

24. The method according to claim 18, wherein the sample is a red blood cell (RBC) sample.

25. The method according to claim 24, wherein the RBC sample is an RBC lysate.
